(19) United States
(12) Patent Application Publication
(10) Pub. No.: US 2016/0010059 A1
(43) Pub. Date: Jan. 14, 2016

(54) EXPANSION OF HEMATOPOIETIC PROGENITOR CELLS BY HISTONE METHYLTRANSFERASE G9a INHIBITION

(71) Applicant: Fernando UGARTE, Santa Cruz, CA (US)

(72) Inventor: Fernando Ugarte, Santa Cruz, CA (US)

(73) Assignee: The Regents of the University of California, Oakland, CA (US)

(21) Appl. No.: 14/773,114

(22) PCT Filed: Mar. 6, 2014
(86) PCT No.: PCT/US14/21422

§ 371 Date: Sep. 4, 2015

Related U.S. Application Data
(60) Provisional application No. 61/773,748, filed on Mar. 6, 2013.

(51) Int. Cl. C12N 5/0789

(52) U.S. Cl. CPC C12N 5/0647; C12N 2501/72

(57) ABSTRACT

The invention provides compositions and methods that may be used to prevent and/or decelerate differentiation of stem cells. The invention may be used to maintain and expand a population of stem cells from a human subject, thereby providing a substantially greater population of stem cells for clinical and therapeutic treatments for various diseases and disorders.

This application claims priority to U.S. Provisional Patent Application Ser. No. 60/773,748 entitled “Expansion of Hematopoietic Progenitor Cells by 69 Inhibition in vitro”, filed 6 Mar., 2013, which is herein incorporated by reference in its entirety for all purposes.

### FIELD OF THE INVENTION

The invention relates to using a histone methyltransferase inhibitor to block or delay the differentiation of, and expand a population of hematopoietic stem cells.

### BACKGROUND

Epigenetic mechanisms evidently play a role in determining the identity of stem cells, but the details of how they contribute are unclear. Hematopoiesis provides an excellent model for understanding the epigenetic regulation of stem cell differentiation. Our initial data clearly demonstrate that global chromatin changes occur upon hematopoietic stem cell (HSC) differentiation into mature blood lineages. Nuclease sensitivity assays revealed that HSC are significantly more sensitive to DNasel digestion than mature cells. In addition, high-resolution electron micrography (EM) and soft X-ray tomography imaging analyses demonstrated a major accumulation of heterochromatin upon HSC differentiation. However, no global chromatin differences are observed between HSC and intermediate progenitors, suggesting that the transition between these early stages of differentiation likely involve more subtle, locus-specific changes.

Epigenetic mechanisms have been shown to play a major role in maintaining stem cell identity, as well as in regulating its cell fate decisions (Barrero et al., 2010; Surani et al., 2007). The importance of epigenetic mechanisms, such as chromatin remodeling, is with no doubt an essential component to our understanding of the function and properties of stem cells (Ho and Crabtree, 2010). Changes in chromatin condensation have direct implications on gene expression and the activation or silencing of entire developmental programs. The recent generation of induced pluripotent stem cells (iPS) further highlights the importance of chromatin remodeling on the process of cell reprogramming (Hochedlinger and Plath, 2009; Koche et al., 2011). Current evidence suggests that open chromatin conformation and the hyperdynamic binding of chromatin structural proteins are key features of embryonic stem cells (ESCs; Meshorer et al., 2006). This is accompanied with an overall transcriptional hyperactivity in ESCs compared to differentiated cells (Efroni et al., 2008). More immature cells also have a higher proportion of DNaseI hypersensitive sites, and the majority of them is lost or exchange upon stem cell differentiation, suggesting major remodeling of their epigenetic landscape (Stergachis et al., 2013). Furthermore, chromatin remodeling proteins such as Chd1 and esBAF, have been shown to be essential to maintain this open chromatin state in ESCs and preserve their self-renewal capacity and pluripotency (Gaspar-Maia et al., 2009; Ho et al., 2009). This open chromatin state of stem cells enables a number of key developmental genes to be in a permissive bivalent conformation, marked by the co-existance of active (H3k4me3) and silencing (H3K27me3) chromatin marks (Azuara et al., 2006; Bernstein et al., 2006). These bivalent genes remain silent, but poised for activation upon the initiation of specific developmental pathways. Overall these observations suggest that chromatin conformation is very dynamic in ESCs and that it changes dramatically upon differentiation, however whether this is also a characteristic of adult stem cells remains unclear.

Hematopoiesis poses as an ideal model for the study of chromatin regulation of pluripotency, since the lineage potential of HSC and intermediate progenitors has been well established (Boyer et al., 2011; Forsberg et al., 2006). Nevertheless, the epigenetic mechanisms underlying the balance between HSC maintenance and their expansion and differentiation into mature hematopoietic are not well understood. Experimental evidence suggests that changes in chromatin structure at specific genomic foci occur during differentiation of HSC, T cells, and red blood cells (Ansel et al., 2006; Attema et al., 2007; de Laat et al., 2008); and that gene expression levels during human HSC differentiation are closely linked to the presence/absence of histone tail modifications (Cui et al., 2009). Genome wide characterization of the epigenetic landscape of various hematopoietic cells populations revealed the presence of bivalent domains not only HSCs, but also in their more differentiated progeny (Adli et al., 2010; Weishaupt et al., 2010). Additionally, chromatin remodeling proteins, such as Bmil, are required to maintain HSC and leukemic stem cell self-renewal capacity, as well as to regulate their lineage specification (Lessard and Sauvageau, 2003; Oguro et al., 2010; Park et al., 2003). On the other hand, leukemic transformation of HSCs is commonly linked to the malfunction of components of the chromatin remodeling machinery, such as the histone H3 lysine 4 (H3K4) methyltransferase MLL (Chen et al., 2010; Krivtsov and Armstrong, 2007). Therefore the elucidation of the underlying epigenetic status of HSC and its dynamics is essential to understand the role of epigenetic modifications in the transition between stages of stem cell differentiation. Here, we show for the first time and at high resolution the global changes in chromatin conformation upon adult stem cell differentiation, and how transition from euchromatin to heterochromatin is essential for proper HSC differentiation.

Accordingly, there is a need in the art to provide compositions and methods to prevent or delay differentiation of stem cells, expand the population of stem cells and that may be used in gene therapy applications and treatments of diseases and disorders of autoimmune disease, neoplasias, and cancers.

### SUMMARY OF THE INVENTION

The invention provides a method and compositions to delay differentiation of hematopoietic stem cells, the method resulting in an at least four-fold enrichment in the c-Kit positive, lineage negative, Sca-1 positive (KLS) fraction of the hematopoietic stem cells.

In one embodiment the invention provides a method for delaying differentiation of hematopoietic stem cells, the method comprising the steps of (i) providing a plurality of hematopoietic stem cells, wherein the hematopoietic stem cells further comprise a KLS fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in delaying differentiation of the hematopoietic stem cells.

In another embodiment, the invention provides a method for maintaining and expanding a population of hematopoietic stem cells, the method comprising the steps of (i) providing a plurality of hematopoietic stem cells, wherein the hematopoietic stem cells further comprise a KLS fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in maintaining and expanding the population of hematopoietic stem cells.

In another embodiment, the invention provide a method for down-regulating expression of a gene in a population of hematopoietic stem cells, the method comprising the steps of (i) providing a plurality of hematopoietic stem cells, wherein the hematopoietic stem cells further comprise a KLS fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in down-regulating a gene in the population of hematopoietic stem cells. In a preferred embodiment, the gene is selected from the group consisting of Egr1, Ndn, Flt3, and Robo4.

In another embodiment, the invention provides a method for up-regulating expression of a gene in a population of hematopoietic stem cells, the method comprising the steps of (i) providing a plurality of hematopoietic stem cells, wherein the hematopoietic stem cells further comprise a KLS fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in up-regulating a gene in the population of hematopoietic stem cells. In one preferred embodiment, the gene is selected from the group consisting of Sox6, Itga2b, Gata1, Hnf4a, Dpp4, Ccr2, Hbb-b1, Itgam, Itgax, Cebpe, Mpo, and Fgf3.

In an alternative embodiment, the method further comprises the steps of (vii) transforming at least one hematopoietic stem cell with a vector, the vector comprising a recombinant nucleic acid; (viii) incubating the transformed hematopoietic stem cell for at least 24 hours; wherein expression of the recombinant nucleic acid corrects a phenotypic defect in the hematopoietic stem cell.

In another embodiment, the invention provides a method for the manufacture of a population of hematopoietic stem cells comprising a recombinant nucleic acid composition, wherein expression of the recombinant nucleic acid composition corrects a phenotypic defect in the population of hematopoietic stem cells for the treatment of a hematopoietic disorder. In another alternative embodiment, the method results in an at least four-fold enrichment of the KLS fraction of the hematopoietic stem cells.

In one preferred embodiment, the hematopoietic stem cells are human hematopoietic stem cells. In one preferred embodiment, the suitable medium is a medium selected from the group consisting of D-MEM, N2B7, M16, F12, Roswell Park Memorial Institute (RPMI), and X-vivo15 media. In a more preferred embodiment, the medium is D-MEM. In one preferred embodiment, the inhibitor of histone methyltransferase is selected from the group consisting of UNC0638, UNC0321, Bix-1294, and Chaetocin. In a more preferred embodiment, the inhibitor of histone methyltransferase is UNC0638. In one preferred embodiment, wherein the suitable temperature is between 30° C. and 42° C. In a more preferred embodiment, the suitable temperature is 37° C. In another preferred embodiment, the incubating period is at least five days.

### BRIEF DESCRIPTION OF THE DRAWINGS

FIG. 1. Nuclear architecture and chromatin conformation during stem cell differentiation. (A) High resolution imaging of embryonic stem (ESC), hematopoietic stem (HSC), multipotent progenitor (MPP), granulocyte macrophage progenitor (GMP), B and granulocyte/macrophage (GM) cells by electron microscopy (left column) and soft X-ray tomography (middle and right columns); (B) Quantification of the ratios of heterochromatin and euchromatin per cell type by EM (left) and SXT (right); (C) Significant positive correlation of the amount of euchromatin and cell size either by EM (left) or SXT (right); (D) Fraction of the volume of heterochromatin associated to the distance to the nuclear envelope show the accumulation of heterochromatin in more differentiated cells; (E) Theoretical model of a cell nucleus as a perfect sphere, and the interphase between hetero- and euchromatin as a perfect disk; (F) Quantification of the total area of hetero/euchromatin interphase by SXT. G) Nuclear folding of ESCs and mature GM cells

FIG. 2. HSPCs chromatin is more accessible to nucleases, but have same frequency of histone post translational modifications (PTMs). (A) HSPCs demonstrate an increase response than mature cells to DNaseI treatment, quantified as the amount of digested DNA at equal concentrations of DNaseI (yellow rectangle), suggesting a more open chromatin structure in HSPCs; (B) micrococcal nuclease (Mnase) sensitivity assays between HSPCs and mature cells; (C) Frequency of histone PTMs does not significantly change between ESCs, HSPCs and more mature cells.

FIG. 3. Distribution of the heterochromatin mark H3K9me3 changes along stem cell differentiation. (A) H3K9me3 IHC demonstrates heterochromatin distribution in the nucleus; (B) Radial distribution of H3K9me3 in embryonic stem (ESC), hematopoietic stem (HSC), multipotent progenitor (MPP), granulocyte macrophage progenitor (GMP), B and T cells (n=12); (C) Quantification of the H3K9me3 radial distribution between the center (inner 50%) and the periphery (both outer 25%) of the nuclei,

FIG. 4. Inhibition of histone methyltransferase G9a impairs HSC differentiation in vitro. (A) FACS plots after 5 days of in vitro culture of mouse HSCs (KLS flk2⁻CD150⁺) with the histone methyltransferase G9a inhibitor UNC0638; (B) Gene expression analysis of a significant number of upregulated HSC-related genes in UNC0638 treated cells compared to the control (DESEQ padj<0.01); (C) ChIP-qPCR demonstrates significantly reduced levels of H3k9me2 on putative G9a targets identified by RNA-seq; (D) UNC0638 treated cells have an increase proliferation capacity after day 5 of in vitro culture, in proportion to the higher ratio of KLS cells; (E) Experimental setup for 24 hrs or 5 days of in vitro culture with and without UNC0638 before transplanting into lethally irradiated mice; (F) Percentage of chimerism of transplanted mice with cells cultured for 24 hrs (top) compared with transplants with cells cultured for 5 days on AFT024 feeder layers (bottom).

FIG. 5. (A) DnaseI sensitivity does not increase significantly in cycling HSCs (KLS flk2−) obtained from mobilized mice with G-CSF. (B) DNA methylation levels are not significantly different between HSPCs and mature cells. (C) Frequency of histone marks in HSCs (KLS flk2−), myeloid progenitors (MP), GM, B and T cells, shows no significant differences between any of these cells populations. Data are means±SEM; n.s. not significant.

FIG. 6. (A) Quantification of the area (by EM) and volumes (by SXT) of hetero- and euchromatin in ESC, HSC MPP, GMP, B and GM cells. (B) Nuclear to cytoplasmic ratio in significantly higher in B cells as well as HSCs and MPPs, as well as the ration of nuclear size to the total cell size. (C) Correlation between nuclear or cell size with the total volumes of heterochromatin or euchromatin.

FIG. 7. (A) Radial distribution of H3K9me3 in ESC, HSC, MPP, GMP, B and T cells. Each line represents the fluorescence intensity of a single cell.

FIG. 8. (A) UNC0638 titration to estimate its optimal concentration for the treatment of HSCs without affecting cell viability. (B) Quantification of total cell and KLS cell numbers under liquid culture conditions with and without UNC0638. (C) Quantification of the amount of H3K9me2 by western blot in UNC0638 treated cells shows that the effect is highly enriched in the KLS fraction. (D) Immunofluorescence for H3K9me3 demonstrate that UNC0638 treated cells have a more disperse distribution of this histone mark compared to the DMSO treated control.

### DETAILED DISCLOSURE OF THE INVENTION

Chromatin remodeling have been shown to be essential for proper stem cell function, however whether global chromatin differences occur in adult stem cell differentiation is not well understood. We used hematopoietic stem cells (HSCs) and their progeny to determine the differences in global chromatin conformation that go along with their differentiation into mature blood cells. Quantification of chromatin composition by high-resolution electron microscopy and soft X-ray tomography demonstrates significant accumulation of heterochromatin in the transition from ESCs into HSPCs, and from HSCPs into mature cells. We also find that hematopoietic stem and progenitor cells (HSPCs) have higher sensitivity to nuclease activity, but similar levels of histone modifications and DNA methylation than fully mature cells. Heterochromatin, marked by H3K9me3, localized preferentially to nuclear periphery for HSCs, multipotent progenitors (MPPs), and mature cells, however granulocyte macrophage progenitor (GMPs) demonstrated a less peripheral and more granular distribution of this mark. Finally, prevention of heterochromatin formation by inhibition of the histone methyl transferase G9a produces a partial delay of HSC differentiation in vitro, by regulating the expression of several factors required for HSC maintenance/differentiation. Overall our results demonstrate that significant global rearrangements on the chromatin structure occur during stem cell differentiation, and that heterochromatin formation by H3K9 methylation is one important mechanism for proper HSC differentiation.

We are currently mapping the epigenetic landscape of HSC, MPP, leukemic HSC, and aged HSC by ChIP-seq using active and repressive histone marks to identify these particular loci, which may identify regulatory elements involved in HSC function. Since heterochromatin accumulation is one of the features of HSC differentiation, we have focused on the role of the histone methyltransferase G9a (G9a), an enzyme that specifically methylates the histone H3 Lys-9 (H3K9) residue and promotes heterochromatin formation. Our data show that inhibition of G9a significantly delays HSC differentiation in vitro, leading to an eight-fold expansion and accumulation of progenitor cells. These in vitro expanded progenitor cells were multipotent, as demonstrated by multilineage readout upon transplantation. These data suggest that heterochromatin formation is necessary for the transition of HSCs into more mature cells. Our goal now is to identify the genomic targets of G9a, to understand the molecular mechanisms that lead to this G9a-mediated delay in HSC differentiation. Overall, our results demonstrate that HSCs have a more open chromatin structure compared to mature hematopoietic cells, and that heterochromatin formation is essential for proper HSC differentiation. Elucidating the underlying chromatin structure of HSCs and its dynamics during differentiation is essential to comprehend the role of chromatin remodeling in the transition between these stages. In addition, comparison to leukemic stem cells will help us understand how epigenetic mechanisms contribute to leukemogenesis.

By blocking or delaying HSC differentiation in vitro, the methods disclosed herein may be used to cultivate and expand HSC from patients for longer periods of time and obtaining more cells for transplantation. In addition, the methods may be used for gene therapy applications that cultivate HSCs ex-vivo for a few days to perform viral transduction with the gene of interest before transplantation into a patient. Use of the methyltransferase inhibitor UNC00638 treatment may maintain HSCs in a more primitive stage during this time, and thus improve the efficiency of the process.

Therefore UNC0638 may be used to delay the differentiation of HSCs and other stem cells such as, but not limited to, ESCs, in vitro.

A more detailed description of the drawings follows.

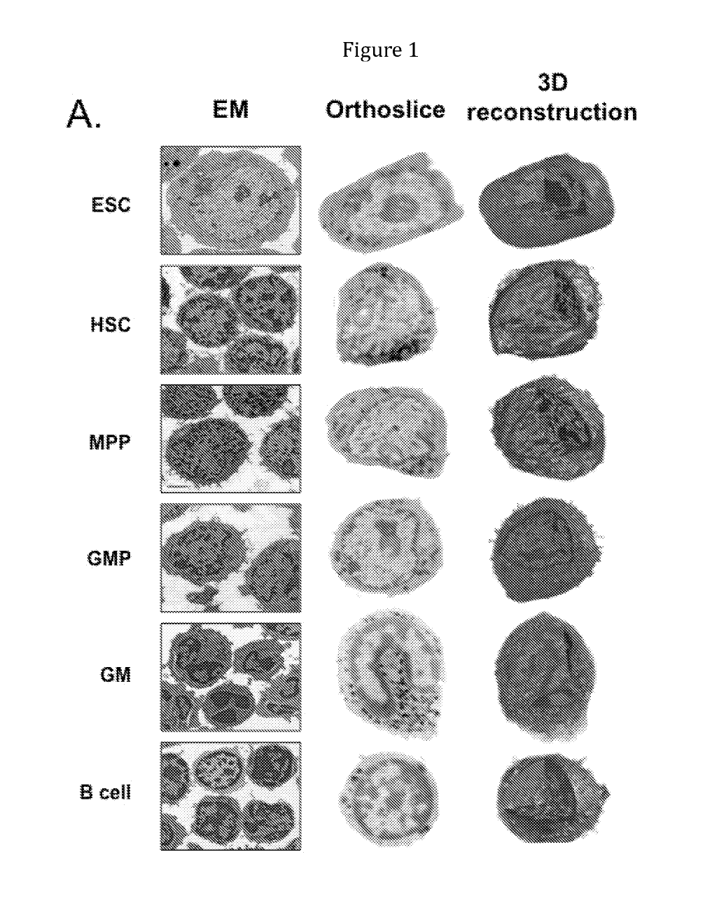

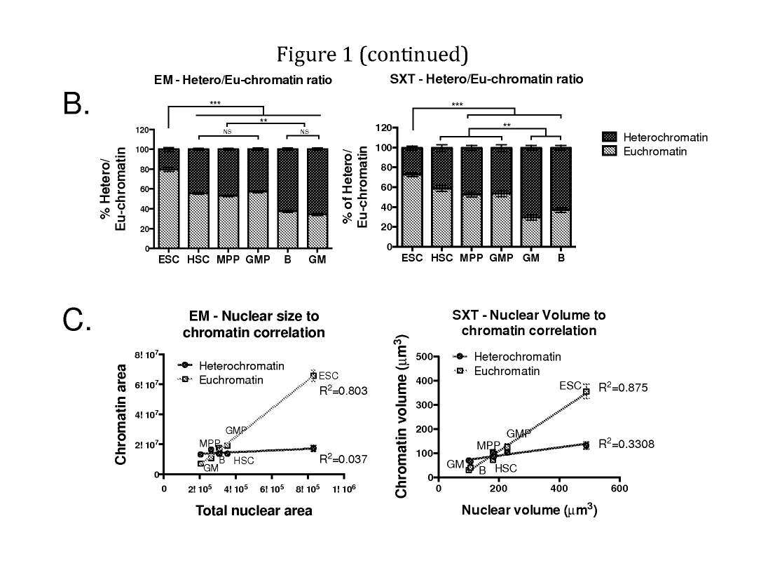

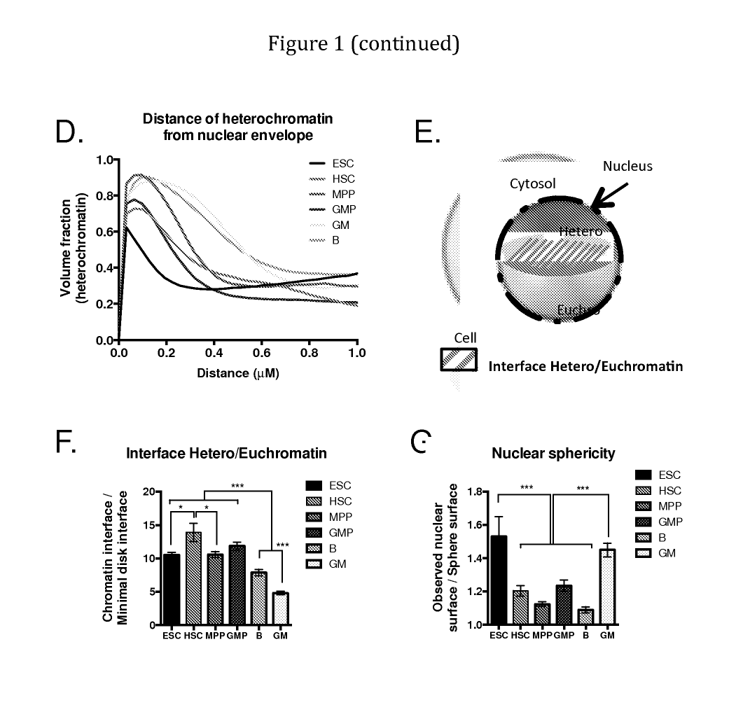

FIG. 1. Nuclear Architecture and Chromatin Conformation During Stem Cell Differentiation.

(A) High resolution imaging of ESCs, HSC, MPP, GMP, B and GM cells by Electron microscopy (left column) and Soft X-ray tomography (middle and right columns) illustrate the different organization of Hetero (blue) and Euchromatin (green) in a single plane as well as in a three-dimensional reconstruction. (B) Quantification of the ratios of heterochromatin and euchromatin per cell type by EM (left) and SXT (right). (EM n=30, SXT n=8). (C) Significant positive correlation of the amount of euchromatin and cell size either by EM (left) or SXT (right). (D) Fraction of the volume of heterochromatin associated to the distance to the nuclear envelope show the accumulation of heterochromatin in more differentiated cells. (E) Theoretical model of a cell nucleus as a perfect sphere, and the interphase between hetero- and eu-chromatin as a perfect disk. (F) Quantification of the total area of hetero/euchromatin interphase by SXT shows that stem and progenitor cells have significantly higher area of interphase than lineage committed cells. Interphase of perfect disk=1. (G) ESCs and mature GM cells have the largest amount of nuclear folding quantified as nuclear sphericity, while HSPCs and B cells are the closest to a theoretical perfect sphere (=1). Data are means±SEM of at least 8 biological replicates; *p<0.05, **p<0.01, ***p<0.001, ns not significant.

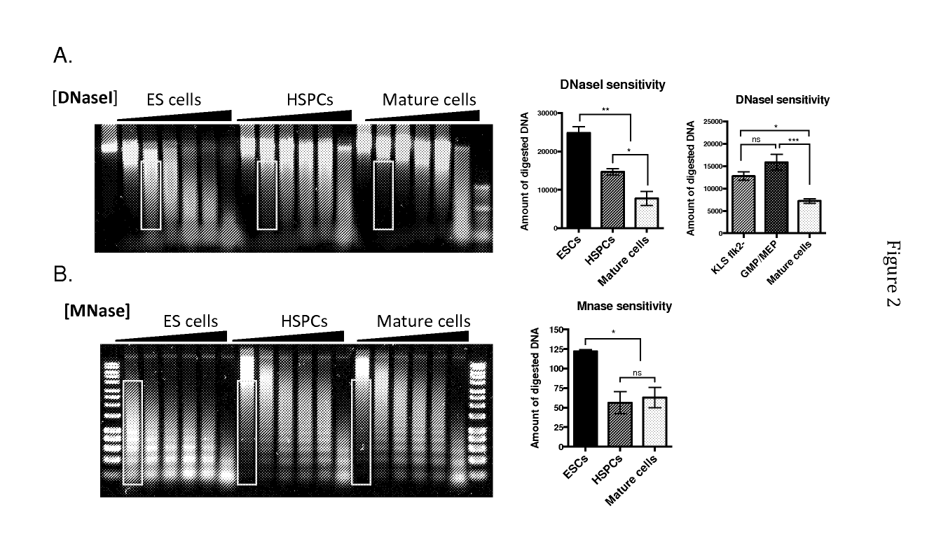

FIG. 2. HSPCs Chromatin is More Accessible to Nucleases, but have Same Frequency of Histone Post Translational Modifications (PTMs).

(A) HSPCs demonstrate an increase response than mature cells to DNaseI treatment, quantified as the amount of digested DNA at equal concentrations of DNaseI (yellow rectangle), suggesting a more open chromatin structure in HSPCs. ESCs cells were used as positive control with higher sensitivity to DNaseI treatment. (B) MNase sensitivity assays shows no differences between HSPCs and mature cells, suggesting that DNaseI differences are not due to differences in linker regions. (C) The frequency of histone PTMs does not significantly change between ESCs, HSPCs and more mature cells, suggesting that the levels of these PTMs do not reflect the differences in global chromatin conformation observed between these cells. Data are means±SEM; *p<0.05, **p<0.01, ***p<0.001, ns not significant.

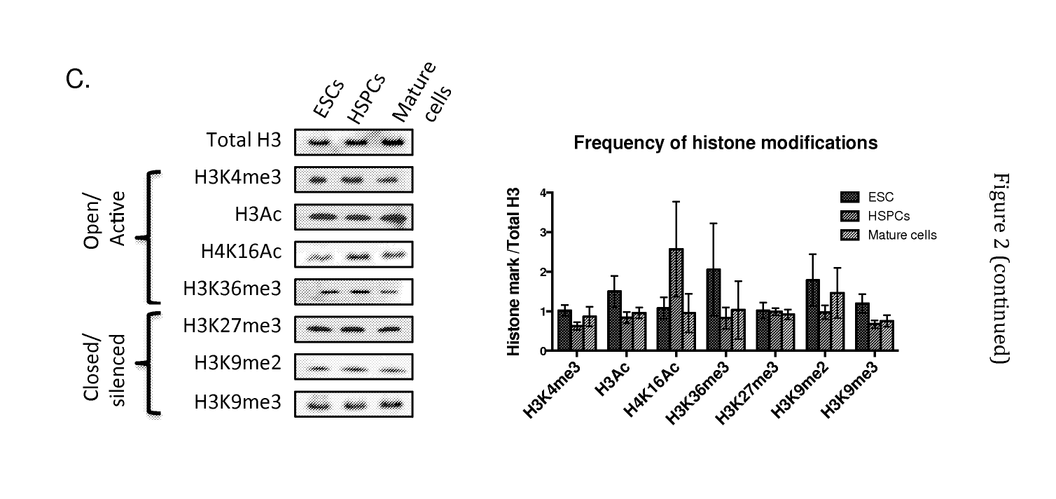

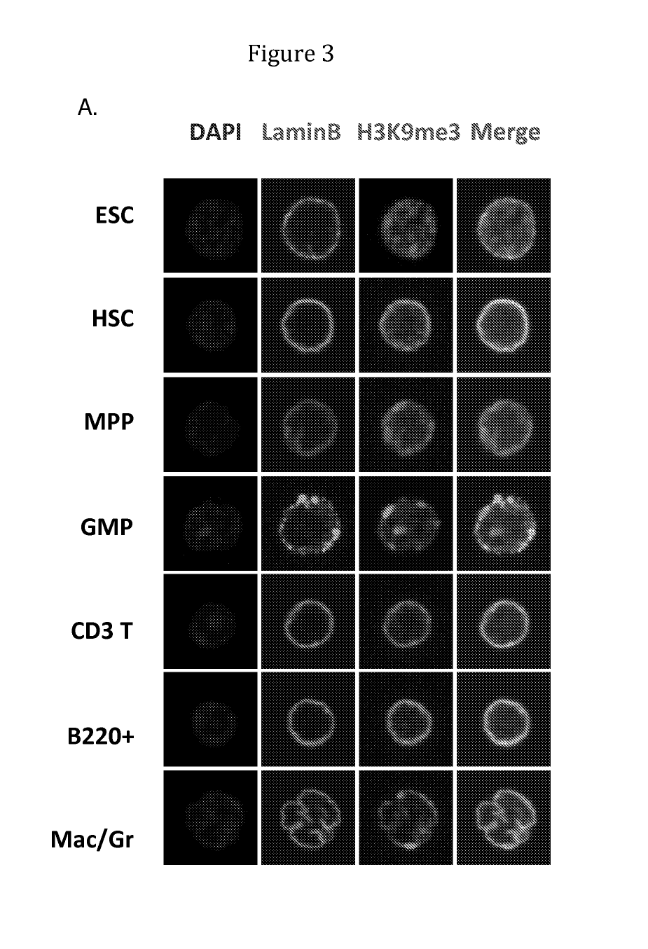

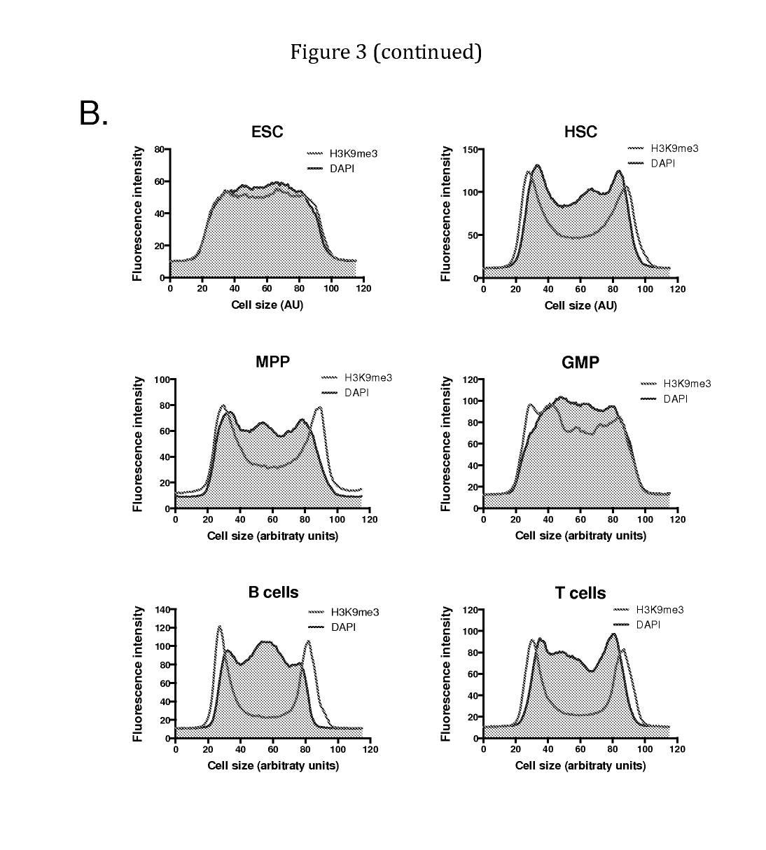

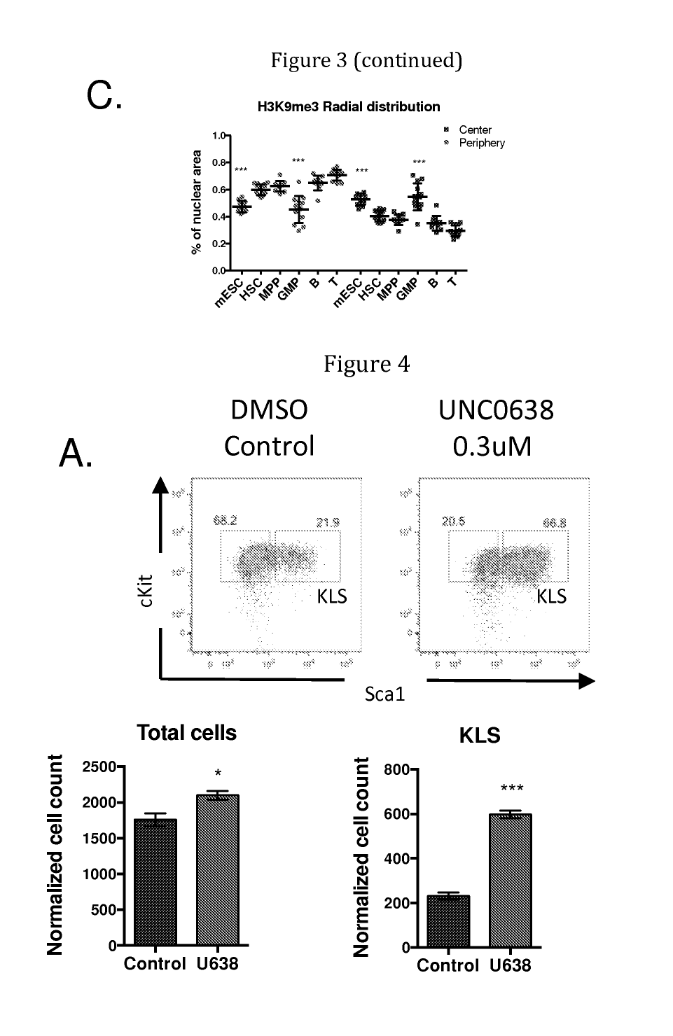

FIG. 3. Distribution of the Heterochromatin Mark H3K9Me3 Changes Along Stem Cell Differentiation.

(A) H3K9me3 IHC demonstrate heterochromatin distribution in the nucleus, going from a more homogeneous distribution in ESCs to a tight pericentric distribution in mature cells. LaminB was used to mark the nuclear envelope. (B) Radial distribution of H3K9me3 in embryonic stem (ESC), hematopoietic stem (HSC), multipotent progenitor (MPP), granulocyte macrophage progenitor (GMP), B and T cells (n=12). GMPs showed a less defined pattern, with significant foci in the center of the nucleus, similarly to ESC. (C) Quantification of the H3K9me3 radial distribution between the center (inner 50%) and the periphery (both outer 25%) of the nuclei, illustrate shift of H3K9me3 from the center to the periphery during stem cell differentiation. Data are means±SEM, *p<0.05, **p<0.01, ***p<0.001, ns not significant.

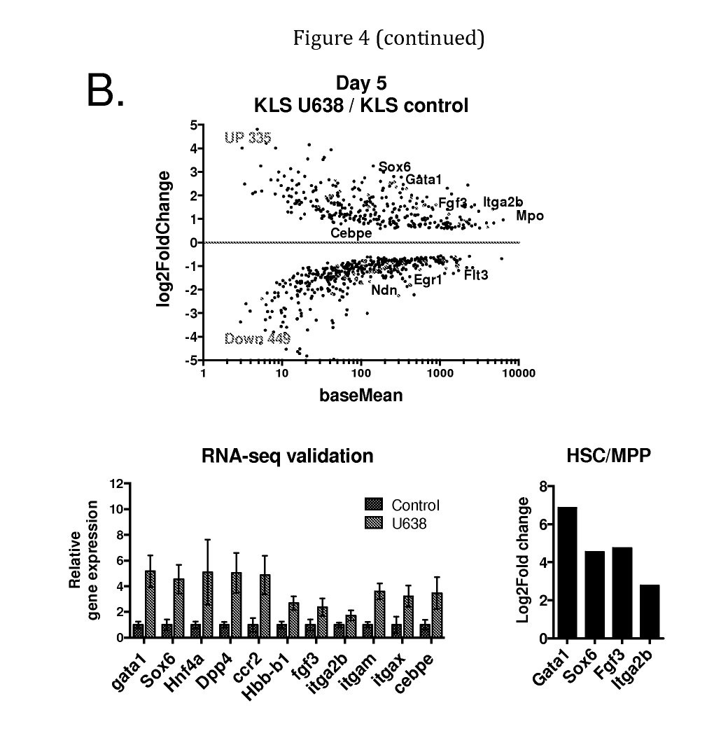

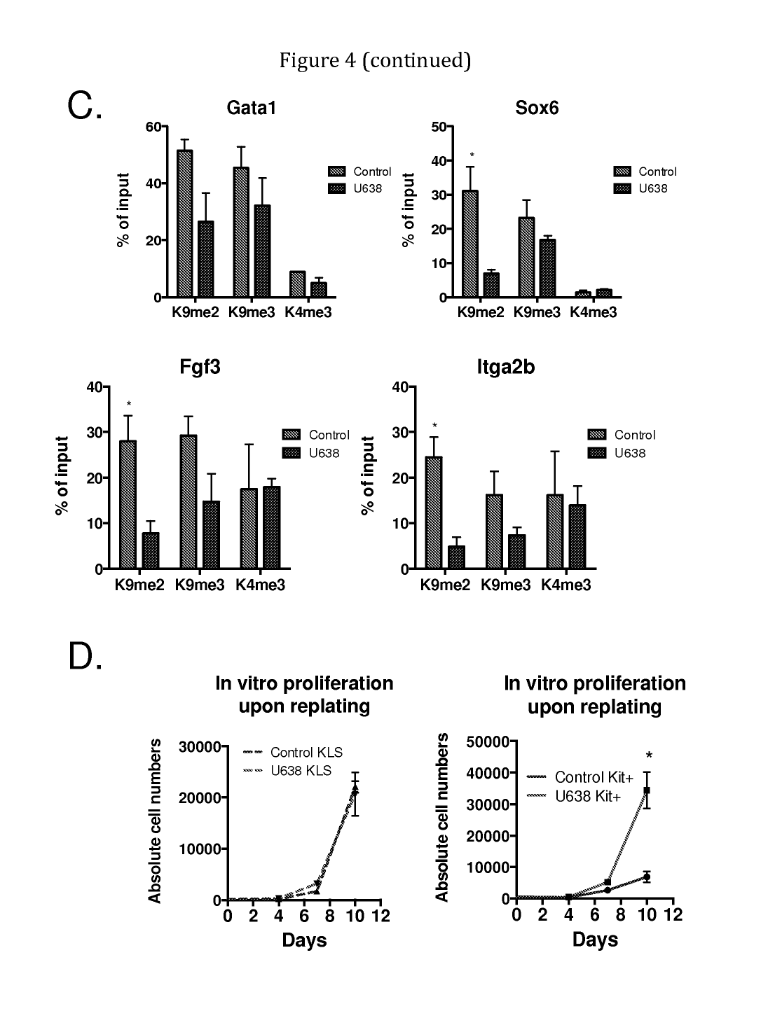

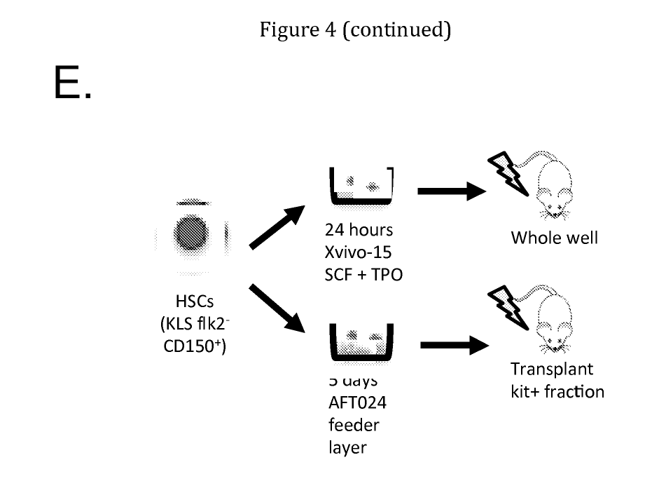

FIG. 4. G9a Inhibition Impairs HSC Differentiation In Vitro.

(A) FACS plots after 5 days of in vitro culture of mouse HSCs (KLS flk2⁻CD150⁺) with the G9a inhibitor UNC0638. A significant increase in the frequency of the KLS is observed in UNC0638 treated cells compared to the DMSO treated control (66.8% vs 21.9%). Quantification of the total number of viable cells and KLS fraction, demonstrate a 5-fold enrichment in KLS cells upon G9a inhibition. (B) Gene expression analysis revealed a significant number of upregulated HSC-related genes in UNC0638 treated cells compared to the control (DESEQ padj<0.01). Some of these genes have higher expression in freshly isolated HSCs compared to MPPs, suggesting that UNC0638 treated cells failed to silence them upon differentiation (n=3). (C) ChIP-qPCR demonstrates significantly reduced levels of H3k9me2 on putative G9a targets identified by RNA-seq. A mild trend toward H3k9me3 reduction is also observed and no differences for the active mark H3K4me3 (n=3). (D) UNC0638 treated cells have an increase proliferation capacity after day 5 of in vitro culture, in proportion to the higher ratio of KLS cells. (E) Experimental setup for 24 hrs or 5 days of in vitro culture with and without UNC0638 before transplanting into lethally irradiated mice. (F) Percentage of chimerism of transplanted mice with cells cultured for 24 hrs (Top) shows a positive trend in higher chimerism for UNC0638 treated cells in all myeloid and lymphoid lineages, though not significant (n=10 per group). On the other hand, transplants with cells cultured for 5 days on AFT024 feeder layers (Bottom), show significantly higher % of GM and B chimerism in UNC0638 treated cells at early time-points, and only a slight trend for other lineages (n=10). Both UNC0638 treated and untreated cells readout in all 5 lineages. Data are means±SEM, *p<0.05, **p<0.01, ***p<0.001, ns not significant.

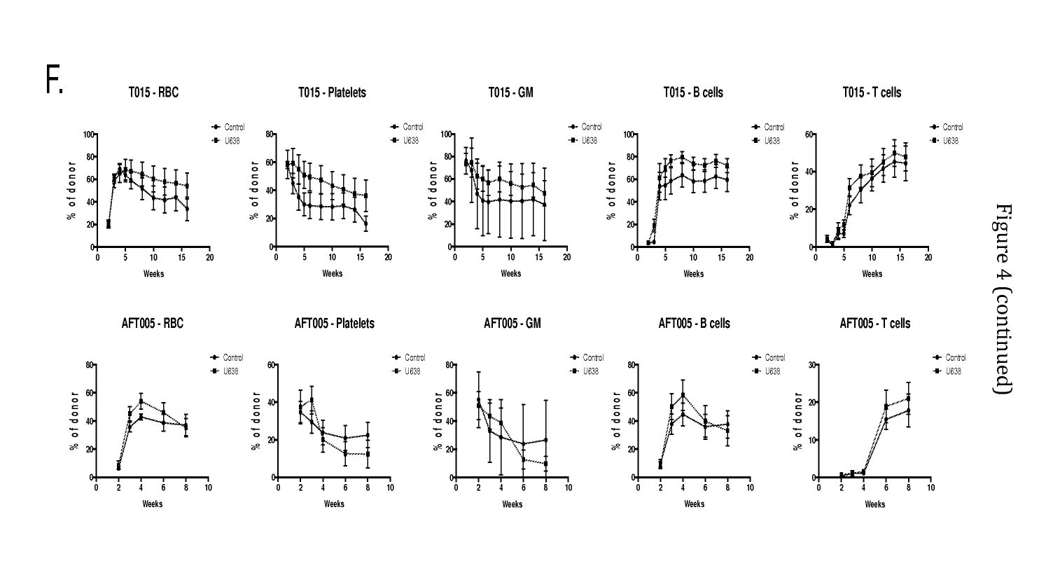

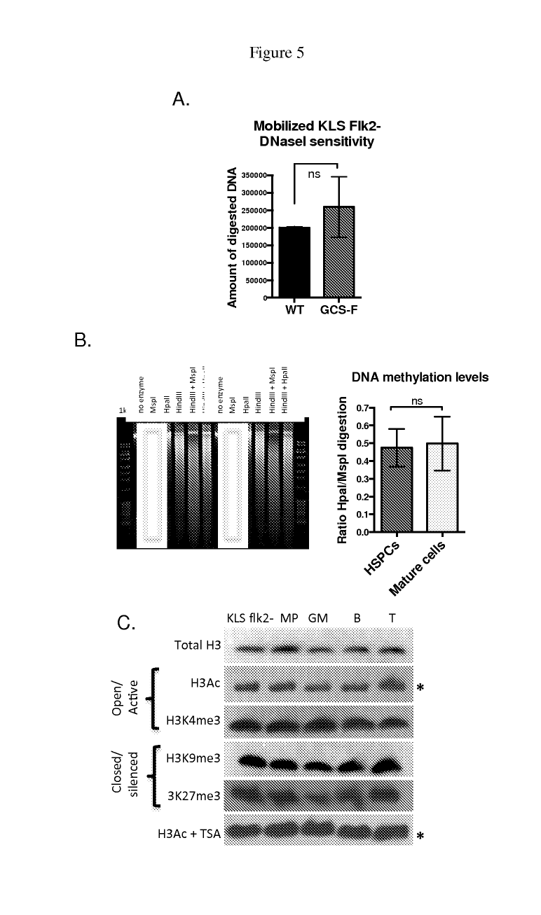

FIG. 5.

(A). DnaseI sensitivity does not increase significantly in cycling HSCs (KLS flk2−) obtained from mobilized mice with G-CSF, suggesting that increase sensitivity in highly proliferative downstream progenitors is not due to their cell cycle status. (B) DNA methylation levels are not significantly different between HSPCs and mature cells, quantified as the relative amount of DNA digestion by the methylation sensitive enzyme HpaII. (C) Frequency of histone marks in HSCs (KLS flk2−), myeloid progenitors (MP), GM, B and T cells, shows no significant differences between any of these cells populations. Data are means±SEM; n.s.: not significant.

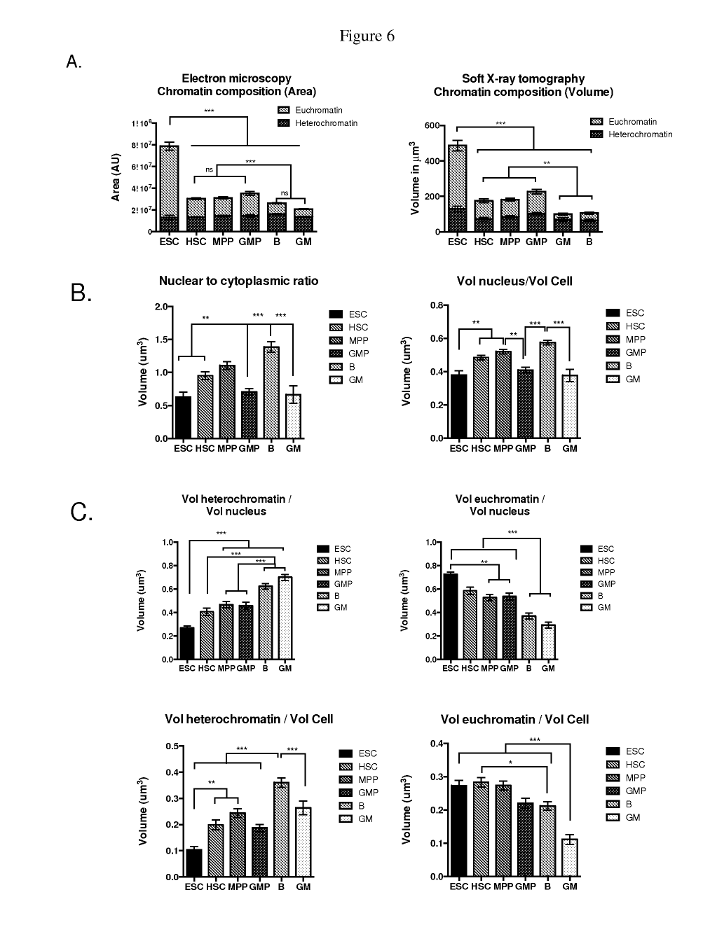

FIG. 6.

(A) Quantification of the area (by EM) and volumes (by SXT) of hetero- and eu-chromatin in ESC, HSC MPP, GMP, B and GM cells. Both analysis demonstrate that the volume of euchromatin is reduced dramatically upon stem cell differentiation, whereas the volume of heterochromatin remains stable. (B) Nuclear to cytoplasmic ratio in significantly higher in B cells as well as HSCs and MPPs, as well as the ration of nuclear size to the total cell size, meaning that in these cells the nucleus accounts for most of the cell volume. (C) Correlation between nuclear or cell size with the total volumes of heterochromatin or euchromatin, demonstrate that nuclear and cell volume gradually changes its composition from euchromatin into heterochromatin.

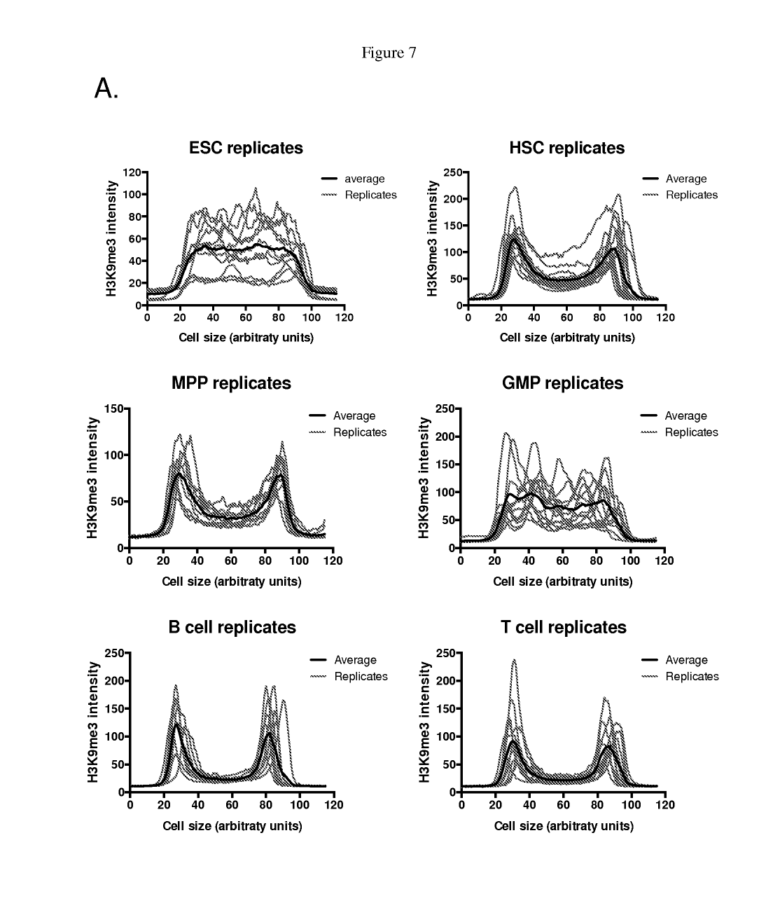

FIG. 7.

(A) Radial distribution of H3K9me3 in ESC, HSC, MPP, GMP, B and T cells. Each line represents the fluorescence intensity of a single cell. Significant differences can be observed between ESCs and GMPs, in which ESCs have a more homogenous distribution across the cells and GMPs have distinct foci of high intensity. The differences between this two cell types can only be precisely observed by looking at individual replicates and not at the average values (FIG. 3b).

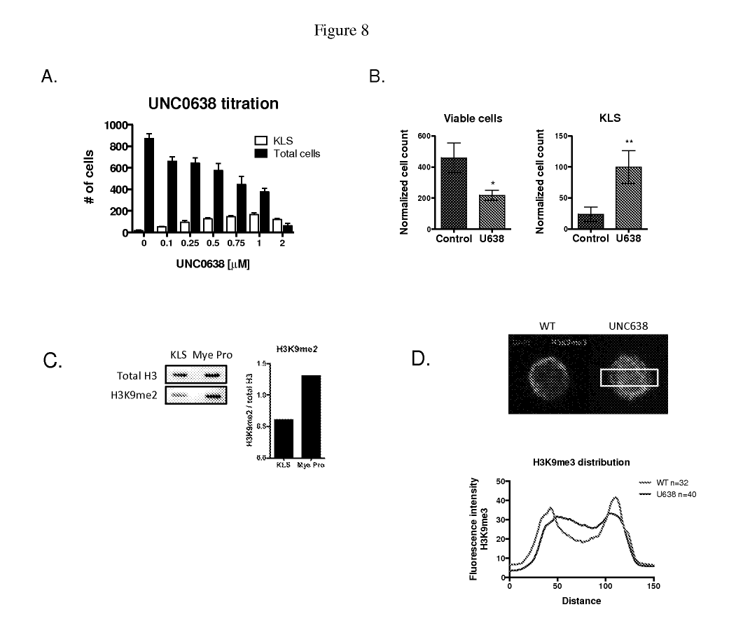

FIG. 8.

(A) UNC0638 titration to estimate its optimal concentration for the treatment of HSCs without affecting cell viability. (B) Quantification of total cell and KLS cell numbers under liquid culture conditions with and without UNC0638 Similarly to the HSCs cultured over AET024 stromal cells, cells in liquid culture only accumulate KLS cells upon treatment with UNC0638, however the total cell number is reduced. (C) Quantification of the amount of H3K9me2 by western blot in UNC0638 treated cells shows that the effect is highly enriched in the KLS fraction. (D) Immunofluorescence for H3K9me3 demonstrate that UNC0638 treated cells have a more disperse distribution of this histone mark compared to the DMSO treated control.

Here, we show for the first time and at high resolution the global changes in chromatin conformation upon adult stem cell differentiation, and how transition from euchromatin to heterochromatin is essential for proper HSC differentiation.

The increasing evidence for the role of chromatin remodeling in the process of stem cell maintenance and differentiation, as well as in cancer development, highlights the importance to clarify the epigenetic mechanisms that still remain unknown (Ho and Crabtree, 2010). Here, we have shown in great detail how the chromatin composition and organization dramatically changes from ESC to hematopoietic stem and progenitor cells, and terminally differentiated blood cells. For the first time we are able to visualize the chromatin organization in the nuclei of rare hematopoietic stem cells isolated from bone marrow. Besides quantifying and characterizing the chromatin composition of various types of stem and progenitor cells of different potential, we also provide new data regarding how the interphase between euchromatin and heterochromatin fractions fluctuates, and how the layer of heterochromatin at the nuclear envelope gets increasingly thicker. We believe that some of these observations open up new questions at how the nuclear architecture of cells is modified in stem cell differentiation.

In general, it has been assumed that chromatin condensation is a gradual process that occurs upon the differentiation of stem cells into a lineage committed cells (Akashi et al., 2003), mainly by looking at both ends of the spectrum, that is ESCs and mature cells (Gaspar-Maia et al., 2011; Meshorer et al., 2006). Here we have expanded this observation by demonstrating for example that at the global level there are no major differences in chromatin composition between adult cells with significantly different self-renewal and lineage potential, such as HSCs, MPPs, or GMPs. Recalling the highly popular concept established by Waddington for the canalization of cell development (Waddington, 1957), our results would suggest that within downhill valleys of development there must be plateau regions where chromatin composition at the global level is not significantly different between closely related cell types. It appears that more importantly than the actual amount of chromatin fractions, it is the spatial distribution of the chromatin fractions that makes the difference between different cell types. The results described herein show that cells such as GMPs are significant outliers in terms of heterochromatin distribution within hematopoietic cells, and not following the expected epigenetic progression. This is highly relevant in the context of cell reprogramming as it has been previously shown that GMPs have a significant advantage in reprogramming efficiency compared to other hematopoietic stem and progenitor cells (Eminli et al., 2009). This has been recently confirmed by Guo et.al. who elegantly demonstrated that GMPs are cells with a privileged position for reprogramming due to their fast cell-cycle kinetics (Guo et al., 2014). The process of cell cycle involves major rearrangements in chromatin structure, therefore it is not surprising that reprogramming efficiency drastically increase in cells with highly dynamic epigenomes (Alabert and Groth, 2012). These observations are certainly important in order to improve the reprogramming efficiency from any type of tissue.

In addition to our observations on the changes in global chromatin conformation, we have also shown how the chromatin remodeler histone methyltransferase G9a plays a significant role in the differentiation of hematopoietic stem cells. Although another group has shown that G9a deletion has minor effects in mouse hematopoiesis (Lehnertz et al., 2010), our results showed that lack of G9a function significantly impair the differentiation abilities of HSCs, by maintaining them in a progenitor cell status (c-Kit positive, lineage negative, Sca-1 positive; KLS) in an in vitro culture setting. These results suggest that G9a function is necessary for the proper silencing of genes associated with the transition of HSCs in to lineage committed cells. We initially expected that the high proportion of stem and progenitor cells upon G9a inhibition would translate in significantly higher readout upon transplantation, however results showed only minor increases in engraftment for these cells. Potential reasons for these results might be that besides “blocking” HSC differentiation, G9a also affects the expression of genes necessary for cell migration and homing (for example, Robo4), or for the proper interaction of HSCs with their niche. Our gene expression data also shows that a significant number of cytoskeleton-associated proteins are significantly down-regulated (FIG. 8e), such as MYH10, which has been recently described as an important component of HSCs for their migration and proliferation capacities (Shin et al., 2013). Interestingly, chromatin condensation appears to directly affect cell migration by regulating nuclear shaping and movement (Gerlitz and Bustin, 2010). Most of the genes activated by G9a inhibition in HSCs are probably in a primed state, ready to be activated or silenced upon differentiation, as HSCs have been shown to contain a large fraction of primed genes associated with a transcriptional promiscuous status (Miyamoto and Akashi, 2005). Also, as shown by Cui et. al., G9a substrate mark H3K9me1 serves as a marker to identify potential enhancers in the genome human HSC, highlighting the importance of this histone mark and its remodeling during HSC development (Cui et al., 2009). Similarly, G9a inhibition in human CD34+ cells also results in an accumulation of this progenitor cell fraction in vitro (Chen et al., 2012). Studies from different stem cell models have additionally shown the significant role of G9a in stem cell differentiation, as G9a regulates the timing of pluripotency gene silencing upon ESC differentiation (Yamamizu et al., 2012), and that G9a inhibitors significantly improve the reprogramming efficiency of adult somatic cells into iPS inhibition for iPSs (Shi et al., 2008). Overall the data presented herein contributes to understand the significance of the transition from euchromatin to heterochromatin in regulating stem and progenitor cell differentiation, not only at specific regulatory genes, but also at the global level. A comprehensive examination on how chromatin remodeling regulates stem cell function is fundamental for the correct use of stem cells for regenerative medicine purposes.

In one embodiment, the invention provides administering the subject hematopoietic stem cells in combination with a pharmaceutically acceptable excipient such as sterile saline or other medium, gelatin, an oil, etc., to form pharmaceutically acceptable compositions. The hematopoietic stem cells may be administered alone or in combination with any convenient carrier, diluent, etc., and such administration may be provided in single or multiple dosages. Useful carriers include solid, semi-solid or liquid media including water and non-toxic organic solvents. The hematopoietic stem cells may be provided in any convenient form for that may be used to administer the hematopoietic stem cells to an individual, for example, as an injectable aqueous or non-aqueous solution or suspension that may be administered to a site of therapy, or administered systematically through an individual's circulatory system. The hematopoietic stem cells may be used, for example, in the treatment of a blood disorder, a cancer, metastasis, hematopoietic transplant, etc. The hematopoietic stem cells may be used, for example, in stem cell therapy, where cells are administered to an individual for the treatment of a tissue disorder. The hematopoietic stem cells may be advantageously combined and/or used in combination with other therapeutic or prophylactic agents, different from the subject hematopoietic stem cells. The hematopoietic stem cells may also be used in in vitro methods for creating stem cell lines, stem cell-derived tissues, and stem cell-derived organs, and the like. In many instances, administration in conjunction with the subject hematopoietic stem cells enhances the efficacy of such agents, see, for example, Goodman & Gilman's The Pharmacological Basis of Therapeutics, 9^{th} Ed., 1996, McGraw-Hill.

Hematopoietic stem cells and/or hematopoietic progenitor cells expanded by the method of the present invention can be used as a cell transplant. Because hematopoietic stem cells can differentiate into blood cells of all lineages, they may be transplanted after differentiated into a certain type of blood cells ex vivo. Hematopoietic stem cells and/or hematopoietic progenitor cells expanded by the method of the present invention may be transplanted as they are, or after enrichment using a cell surface antigen as an index, for example, by a magnetic bead method or by a cell sorting method. Such a cell surface antigen molecule may be CD2, CD3, CD4, CD8, CD13, CD14, CD15, CD16, CD19, CD24, CD33, CD34, CD38, CD41, CD45, CD56, CD66, CD90, CD133, or glycophorin A, but is not restricted thereto. The expanded hematopoietic stem cells and/or hematopoietic progenitor cells may be transplanted to its donor or another individual.

Namely, hematopoietic stem cells and/or hematopoietic progenitor cells expanded by the method of the present invention can be used as a graft for hematopoietic stem cell therapy as a substitute for conventional bone marrow or cord blood transplantation. The transplantation of hematopoietic stem cells and hematopoietic progenitor cells expanded by the method of the present invention is carried out in the same manner as conventional bone marrow or cord blood transplantation, except for the cells to be used. Hematopoietic stem cells and/or hematopoietic progenitor cells expanded by the method of the present invention can also be used as a graft to promote regeneration of nerves and muscles damaged by a traumatic injury or a vascular disorder. The graft may be a composition containing a buffer solution, an antibiotic, a pharmaceutical in addition to hematopoietic stem cells and/or hematopoietic progenitor cells expanded by the method of the present invention.

The hematopoietic stem cell and/or hematopoietic progenitor cell transplant obtained by expansion by the method of the present invention is useful for treatment of not only various types of leukemia but also various diseases. For example, in a case of treatment of a solid cancer patient by chemotherapy or radiotherapy which may cause myelosuppression as a side effect, the patient can recover from hematopoietic damage quickly if the hematopoietic stem cells and/or hematopoietic progenitor cells collected from the bone marrow or peripheral blood of the patient preliminarily to the treatment are expanded ex vivo and returned to the patient after the treatment. Thus, a more intense chemotherapy becomes available with an improved therapeutic effect. It is also possible to alleviate a deficiency in a certain type of blood cells in a patient by differentiating hematopoietic stem cells and/or hematopoietic progenitor cells obtained by the method of the present invention into such a type of blood cells and returning them into the patient. A transplant obtained by the method of the present invention is effective against diseases accompanying decrease in hematopoietic cells and/or hematopoietic insufficiency, diseases accompanying increase in hematopoietic cells, diseases accompanying hematopoietic dysfunction, decrease in immunocytes, increase in immunocytes, diseases accompanying autoimmunity, immune dysfunction, diseases accompanying nerve damage, diseases accompanying muscle damage and ischemic diseases. As specific examples, chronic granulomatosis, severe combined immunodeficiency syndrome, adenosine deaminase (ADA) deficiency, agammaglobulinemia, Wiskott-Aldrich syndrome, Chediak-Higashi syndrome, immunodeficiency syndrome such as acquired immunodeficiency syndrome (AIDS), C3 deficiency, congenital anemia such as thalassemia, hemolytic anemia due to enzyme deficiency and sicklemia, lysosomal storage disease such as Gaucher's disease and mucopolysaccharidosis, adrenoleukodystrophy, various kinds of cancers and tumors, especially blood cancers such as acute or chronic leukemia, Fanconi syndrome, aplastic anemia, gramulocytopenia, lymphopenia, thrombocytopenia, idiopathic thrombocytopenic purpura, thrombotic thrombocytopenic purpura, Kasabach-Merritt syndrome, malignant lymphoma, Hodgkin's disease, chronic hepatopathy, renal failure, massive blood transfusion of bank blood or during operation, hepatitis B, hepatitis C, severe infections, systemic lupus erythematodes, articular rheumatism, xerodermosteosis, systemic sclerosis, polymyositis, dermatomyositis, mixed connective tissue disease, polyarteritis nodosa, Hashimoto's disease, Basedow's disease, myasthenia gravis, insulin dependent diabetes mellitus, autoimmune hemolytic anemia, snake bite, hemolytic uremic syndrome, hypersplenism, bleeding, Bernard-Soulier syndrome, Glanzmann's thrombasthenia, uremia, myelodysplastic syndrome, polycythemia rubra vera, erythremia, essential thrombocythemia, myeloproliferative disease, traumatic spinal cord injury, nerve injury, neurotmesis, skeletal muscle injury, scarring, diabetes mellitus, cerebral infarction, myocardial infarction, obstructive arteriosclerosis and the like may be mentioned.

Hematopoietic stem cells expanded according to the present invention can be used for gene therapy. Gene therapy using hematopoietic stem cells has been difficult because the transfer of a target gene into hematopoietic stem cells at the stationary phase is inefficient, and hematopoietic stem cells differentiate in culture during a gene transfer procedure. However, use of the low-molecular-weight compounds of the present invention in culture makes it possible to expand hematopoietic stem cells while suppressing differentiation of hematopoietic stem cells and improve the gene transfer efficiency considerably. In the gene therapy, a therapeutic gene is transfected into hematopoietic stem cells using the low-molecular-weight compounds of the present invention, and the resulting transfected cells (that is, transformed hematopoietic stem cells) are transplanted into patients. The therapeutic gene to be transfected is appropriately selected among genes for hormones, cytokines, receptors, enzymes, polypeptides and the like according to the disease (Advance in Pharmacology 40, Academic Press, 1997). Specific examples of the gene include genes for insulin, amylase, proteases, lipases, trypsinogen, chymotrypsinogen, carboxypeptidases, ribonucleases, deoxyribonucleases, phospholipase A2, esterases, α1-antitrypsin, blood coagulation factors (VII, VIII, IX and the like), protein C, protein S, antithrombin, UDP glucuronyl transferase, ornithine transcarbanoylase, hemoglobin, NADPH oxidase, glucocerebrosidase, α-galactosidase, α-glucosidase, α-iduronidase, chytochrome P450 enzymes, adenosine deaminase, Bruton kinase, complements C1 to C4, JAK3, common cytokine receptor γ chain, Ataxia Telangiectasia Mutated (ATM), Cystic Fibrosis (CF), myocilin, thymic humoral factor, thymopoietin, gastrin, selectins, cholecystokinin, serotinin, substance P, Major Histocompatibility Complex (MHC), multiple drug resistance factor (MDR-1), and the like.

In addition, RNA genes suppressing expression of disease genes are effective as therapeutic genes and can be used in the method of the present invention. For example, antisense RNA, siRNA, shRNA decoy RNA, ribozymes and the like may be mentioned.

For transfer of a therapeutic gene into hematopoietic stem cells, ordinary gene transfer methods for animal cells, such as those using vectors for animal cells such as retrovirus vectors like murine stem cell vector (MSCV) and Moloney murine leukemia virus (MmoLV), adenovirus vectors, adeno-associated virus (AAV) vectors, herpes simplex virus vectors and lentivirus vectors (for vectors for gene therapy, see Verma, I. M., Nature, 389:239, 1997), calcium phosphate coprecipitation, DEAE-dextran transfection, electroporation, a liposome method, lipofection, microinjection or the like may be used. Among them, retrovirus vectors, adeno-associated virus vectors or lentivirus vectors are preferred because their integration into the chromosomal DNA is expected to allow eternal expression of the gene.

For example, an adeno-associated virus (AAV) vector is prepared as follows. First, 293 cells are transfected with a vector plasmid obtained by inserting a therapeutic gene between the ITRs (inverted terminal repeats) at both ends of wild-type adeno-associated virus DNA and a helper plasmid for supplementing virus proteins and subsequently infected with an adenovirus as a helper virus to induce production of virus particles containing AAV vectors. Instead of the adenovirus, a plasmid for expression of an adenovirus gene that functions as a helper may be transfected. Next, hematopoietic stem cells are infected with the virus particles. It is preferred to insert an appropriate promoter, enhancer, insulator or the like upstream of the target gene in the vector DNA to regulate expression of the gene. Introduction of a marker gene such as a drug resistance gene in addition to the therapeutic gene makes it easy to select cells carrying the therapeutic gene. The therapeutic gene may be a sense gene or an antisense gene.

When hematopoietic stem cells are transfected with a therapeutic gene, the cells are cultured by an appropriate method selected from the culture methods mentioned above for expansion of hematopoietic stem cells by the person in charge. The gene transfer efficiency can be evaluated by a standard method in the art. It is possible to transfect a gene into hematopoietic stem cells otherwise, expand the resulting cells (transformed hematopoietic stem cells) by the above-mentioned method of expanding hematopoietic stem cells and use the resulting transformed hematopoietic stem cells for gene therapy.

The transplant for gene therapy may be a composition containing a buffer solution, an antibiotic, a pharmaceutical and the like in addition to transformed hematopoietic stem cells. The diseases to be treated by gene therapy targeting blood cells include chronic granulomatosis, severe combined immunodeficiency syndrome, adenosine deaminase (ADA) deficiency, agammaglobulinemia, Wiskott-Aldrich syndrome, Chediak-Higashi syndrome, immunodeficiency syndrome such as acquired immunodeficiency syndrome (AIDS), hepatitis B, hepatitis C, congenital anemia such as thalassemia, hemolytic anemia due to enzyme deficiency, Fanconi's anemia and sicklemia, lysosomal storage disease such as Gaucher's disease and mucopolysaccharidosis, adrenoleukodystrophy, various kinds of cancers and tumors.

Expanded or transfected hematopoietic stem cells and/or hematopoietic progenitor cells may be infused by drip, for example, in the case of treatment of leukemia, into patients pretreated with an anticancer drug, total body irradiation or an immunosuppressive drug for eradication of cancer cells or for facilitation of donor cell engraftment. In such cases, the disease to be treated, the pretreatment and the cell transplantation method are selected appropriately by the person in charge. The engraftment of transplanted hematopoietic stem cells and/or hematopoietic progenitor cells in the recipient, the recovery of hematopoiesis, the presence of side effects of the transplantation and the therapeutic effect of the transplantation can be judged by an ordinary assay used in transplantation therapy.

As described above, the present invention makes it possible to expand hematopoietic stem cells and/or hematopoietic progenitor cells and to carry out transplantation therapy and gene therapy safely and easily in a short term by using the expanded cells. Because hematopoietic stem cells and/or hematopoietic progenitor cells can be expanded efficiently by the method of the present invention, the specific compounds of the present invention can be used as a reagent for research on hematopoietic stem cells and/or hematopoietic progenitor cells. For example, in a study to elucidate the factor regulating differentiation and growth of hematopoietic stem cells by identifying the colony forming cells in a culture of hematopoietic stem cells and analyzing the change in cell surface differentiation markers and gene expression, when hematopoietic stem cells are cultured in the presence of a putative factor, addition of a compound of the present invention makes it possible to expand the hematopoietic stem cells and/or hematopoietic progenitor cells to be analyzed efficiently. The incubation conditions, the incubator and the culture medium, the species and amount of the compound of the present invention, the kinds and amounts of additives and the incubation time and temperature used to elucidate such a factor may be selected appropriately from those disclosed herein by the person in charge. The colony forming cells emerging in the culture can be observed under a microscope normally used in the art, optionally after staining them using an antibody specific for the colony forming cells. The change in gene expression caused by such a putative factor can be detected by analyzing DNA or RNA extracted from the cells by southern blotting, northern blotting, RT-PCR or the like. The cell surface differentiation markers can be detected by ELISA or flow cytometry using a specific antibody to examine the effect of the putative factor on differentiation and growth of the cells.

### Replacement of Stressed or Dead Cells

A method for replacing cells that have been damaged or killed due to an abnormal condition or stress is also provided by applying the method for increasing the characteristic of HSC. These damaged or dead cells may be blood cells of the lymphoid, myeloid, or erythroid lineages. These damaged or dead cells may be erythrocytes (red blood cells), platelets, granulocytes (such as neutrophils, basophils, and eosinophils), macrophages, B-lymphocytes, T-lymphocytes, or Natural killer cells. These damaged or dead cells may be differentiated cells such as muscle (skeletal myocytes and cardiomyocytes), brain, liver, skin, lung, kidney, intestinal, or pancreatic.

Representative examples of conditions, stresses or treatments that can cause cell damage or death may include cancer treatments, for example, radiation therapy or chemotherapy; temperature shock; exposure to harmful doses of radiation, for example, workers in nuclear power plants, the defense industry or radiopharmaceutical production, or duties of a soldiers; cell aging; wounding; poisoning; chemical or heat burns, viral infections, and bacterial infection.

The method replacing damaged or killed cells may be accomplished by administering to a patient in needed thereof through an autologous or heterologous treatment regimen. A return of the patient's own stem cells or a donor's stem cells to the patient may supplement or repopulate the patient's pool of HSC. The composition may be administered to the patient before or after the patient's cells have been damaged or killed. For example, the patient may be treated with the composition, and the HSC may be isolated after a time from the patient. The patient may then be exposed to the injury, stress or condition. The patient may subsequently be administered his/her own isolated HSC. Similarly, the method may be accomplished through a donor. The donor may be administered the composition. After a time, the HSC may be isolated from the donor and administered to the patient.

### Treatment of Cells Damaged or Killed During Cancer Treatment

The abnormal condition may be damage to normal tissue and cells attributable to cancer or cancer treatments. For example, the method may be used for providing a supply of stem cells to a patient undergoing chemotherapy. Cancer treatment to eradicate a patient's cancer cell population may eliminate the patient's bone marrow stem cells. A return of the patient's own or a donor's stored stem cells to the patient may supplement or repopulate the patient's in vivo pool of HSCs. The method may increase the number of HSC and mobilize these cells from the bone marrow to the bloodstream and may allow the use of greater doses of cancer treatments such as chemo- or radiotherapy, but with less risk than bone marrow transplantation.

### (1) Autologous or Heterologous Stem Cell Population Transplant

The composition may be administered to a patient before undergoing cancer treatment or to a donor. Blood or peripheral white blood cells (PWBC), which may comprise a stem cell population comprising HSC, may be isolated from the patient or donor. The cells may be isolated from the patient after administering the composition and prior to cancer treatment. The autologous or heterologous stem cell population may be stored for future use. The stem cell population may later be administered to the patient who has previously undergone a cancer treatment. In addition, the stored autologous stem cells may be used in transplants. The composition may used in hematological bone marrow stem cell transplantation. The composition may enhance the success of transplantation before, during, and following immunosuppressive treatments. Autologous stem cell transplants may have the advantage of a lower risk of graft rejection and infection, since the recovery of immune function may be efficient. The incidence of a patient experiencing graft-versus-host disease may be very low as the donor and recipient patients are the same individual.

### (2) Chemotherapy

The cancer treatment may comprise administration of a cytotoxic agent or cytostatic agent, or combination thereof. The cytotoxic agent may prevent cancer cells from multiplying by: (1) interfering with the cell's ability to replicate DNA and (2) inducing cell death and/or apoptosis in the cancer cells. The cytostatic agent act via modulating, interfering or inhibiting the processes of cellular signal transduction that regulate cell proliferation.

Classes of compounds that may be used as cytotoxic agents include the following: alkylating agents (including, without limitation, nitrogen mustards, ethylenimine derivatives, alkyl sulfonates, nitrosoureas and triazenes): uracil mustard, chlormethine, cyclophosphamide (CYTOXAN), ifosfamide, melphalan, chlorambucil, pipobroman, triethylene-melamine, triethylenethiophosphoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, bleomycin, dactinomycin, daunorubicin, doxorubicin, epirubicin, idarubicin, ara-c, paclitaxel (paclitaxel is commercially available as TAXOL), mithramycin, deoxyco-formycin, mitomycin-c, 1-asparaginase, interferons (preferably IFN-α), etoposide, and teniposide.

Other proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their cytotoxic activity. Microtubule affecting agents that may be used include, but are not limited to, allocolchicine (NSC 406042), halichondrin B (NSC 609395), colchicine (NSC 757), colchicine derivatives (for example, NSC 33410), dolastatin 10 (NSC 376128), maytansine (NSC 153858), rhizoxin (NSC 332598), paclitaxel (TAXOL, NSC 125973), TAXOL derivatives (for example, derivatives (for example, NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; and Panda (1996) J. Biol. Chem 271:29807-29812.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Cytostatic agents that may be used also hormones and steroids (including synthetic analogs): 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, and zoladex.

Other cytostatic agents are antiangiogenics, such as matrix metalloproteinase inhibitors, and other VEGF inhibitors, such as anti-VEGF antibodies and small molecules such as ZD6474 and SU6668 are also included. Anti-Her2 antibodies from Genentech may also be utilized. A suitable EGFR inhibitor is EKB-569 (an irreversible inhibitor). Also included are Imclone antibody C225 immuno specific for the EGFR, and src inhibitors. Also suitable for use as a cytostatic agent is CASODEX (bicalutamide, Astra Zeneca) that renders androgen-dependent carcinomas non-proliferative. Yet another example of a cytostatic agent is the antiestrogen TAMOXIFEN that inhibits the proliferation or growth of estrogen dependent breast cancer Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 inhibitors, Src kinase inhibitors, and PDGF inhibitors.

### (3) Radiation Therapy for Cancer

The cancer treatment may comprise radiation therapy. The radiation therapy may be external beam radiation, internal radiation therapy, or conformal radiation therapy, in which a computer is used to shape the beam of radiation to match the shape of the tumor. The radiation used in radiation therapy may come from a variety of sources, including an x-ray, electron beam, or gamma rays. The doses and timing of administration of the radiation during radiation therapy can and will vary depending on the location and extent of the cancer. The composition may be administered with a radioprotectant during radiation therapy, as described above.

Chemotherapy/Radiation and/or Antiviral Therapy for Treating HIV Infection

The method may comprise administering a composition that increases the number of HSC in bone marrow and mobilizes these cells to the bloodstream to alleviate or treat the symptoms of chemotherapy or radiation therapy associated with treatment of an immune deficiency. The composition may be administered to a patient who has undergone myeloablative chemotherapy or radiotherapy for AIDS. For example, the composition may be administered to a patient to mobilize or increase the number of HSC from a patient's bone marrow. The mobilized HSC may then be collected from peripheral blood by leukapheresis. HSC may then enriched from the collected peripheralized blood by immunoadsorption using anti-CD34 antibodies. Optionally, the enriched HSC may be expanded ex vivo by culturing them in the presence of agents that stimulate proliferation of stem cells. Following administration of myeloablative chemotherapy or radiotherapy, the enriched, and optionally expanded HSC may then be returned to the patient's circulating blood and allowed to engraft themselves into the bone marrow.

In addition, this method further optionally involves administration to the patient of anti-HIV compounds, such as antivirals such as AZT, soluble CD4, and CD4-directed blockers of the AIDS virus or antisense or antigene oligonucleotides, both before and after the return of the enriched and optionally expanded HSC to the patient's circulating blood. This step serves a “mopping up” function to prevent residual virus from infecting the progeny of the newly returned stem cells.

### Modulation of Cell Aging

The method described herein may increase the number of HSC and mobilize these cells to the bloodstream. Autologous stem cells may be isolated from blood or peripheral white blood cells and administered for replacing stressed or dead cells due to cell aging.

### Radiation

The method described herein may replace stressed or dead cells attributable to radiation exposure. The method may also replace stressed or damaged cells due to radiation therapy.

Exposure to ionizing radiation (IR) may be short- or long-term, it may be applied as a single dose or multiple doses, to the whole body or locally. Thus, nuclear accidents or military attacks may involve exposure to a single high dose of whole body irradiation (sometimes followed by a long-term poisoning with radioactive isotopes). Likewise, a single dose of radiation is generally used for the pretreatment of bone marrow transplant patients when it is necessary to prepare the host's hematopoietic organs for the donor's bone marrow by “cleaning” them from the host blood precursors.

At the molecular and cellular level, radiation particles may lead to breakage in the DNA and cross-linking between DNA, proteins, cell membranes and other macromolecular structures. Ionizing radiation may also induce secondary damage to the cellular components by giving rise to free radicals and reactive oxygen species (ROS). Multiple repair systems counteract this damage, such as several DNA repair pathways that restore the integrity and fidelity of the DNA, and antioxidant chemicals and enzymes that scavenge the free radicals and ROS and reduce the oxidized proteins and lipids. Cellular checkpoint systems are present to detect the DNA defects and delay cell cycle progression until the damage is repaired or a decision to commit the cell to growth arrest or programmed cell death (apoptosis) is reached.

At the organism level, the immediate effects of low and moderate levels of radiation are largely caused by cell death, which leads to radiation-induced inflammation. At higher radiation levels, the so-called hematopoietic and gastrointestinal syndromes lead to short-term radiation-induced death. The hematopoietic syndrome is characterized by the loss of hematopoietic cells and their progenitors, thereby making it impossible to regenerate blood and the lymphoid system. Death usually occurs as a consequence of infection (due to immunosuppression), hemorrhage and/or anemia. The gastrointestinal syndrome is characterized by massive cell death in the intestinal epithelium, predominantly in the small intestine, followed by the disintegration of the intestinal wall and death from bacteriemia and sepsis. The hematopoietic syndrome manifests itself at lower doses of radiation and leads to a more delayed death than the gastrointestinal syndrome. Very high doses of radiation can cause nearly instant death by eliciting neuronal degeneration.

The method described herein may also increase the scale of protection from ionizing radiation.

### Burn Treatment

The method may comprise administering a composition that increases the number of HSC in bone marrow and mobilizes these cells to the bloodstream to alleviate or treat a burn. The method may comprise increasing a characteristic of a HSC population and isolating peripheral white blood cells. The peripheral white blood cells may comprise autologous stem cells, which may be administered to a burned mammal. The autologous stem cells may be a source of trophic factors that promote tissue regeneration at the burn. The burn may be associated with exposure to radiation.

### Treatment of Diseases

A method for treating a disease with HSC is also provided. The method may comprise administering a composition that increases the number of HSC in bone marrow and mobilizes these cells to the bloodstream to replace diseased cells.

a. Repopulating Hemopoietic Cell Pools

The composition may be used in a method of treating a disease or condition for which repopulating the erythroid, myeloid, or lymphoid hematopoietic cell pool is indicated. For example, the composition may be used in a method of treating a disease or condition when HSC transplantation such as bone marrow transplantation into an animal or human is indicated. The composition may be used to restore or prevent a deficiency in hematopoietic cell number in a subject. The deficiency may arise, for example, from a benign disease or condition, genetic abnormality, disease, stress, chemotherapy, or from radiation treatment. This method involves the same steps as described for transplanting mobilized HSC into a patient who has undergone chemotherapy or radiotherapy for AIDS as described above.

b. Cancer Treatment

The method may comprise administering a composition that increases the number of HSC in bone marrow and mobilize these cells to migrate to the bloodstream to alleviate or treat cancer. The cancer may be a hematologic malignancy including, without limitation, hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia. Other cancers that may be treated include the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, larynx, pancreas (including exocrine pancreatic carcinoma), mouth, pharynx, esophagus, stomach, small intestine, colon, rectum, gall bladder, cervix, thyroid, and skin (including squamous cell carcinoma); tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

c. Blood Disorders/Autoimmune Disease

The method may comprise administering a composition that increases the number of HSC in bone marrow and mobilizes these cells to the bloodstream to alleviate or treat the symptoms of a benign disease or condition. The benign disease or disorder may be associated with the hematopoietic system including, without limitation, a hemoglobinopathy, a bone marrow failure syndrome, an immune deficiency, a metabolic/storage disease, a neutrophil disorder, a platelet disease, a viral infection such as an HIV infection, and an autoimmune disorder. The hemoglobinopathy may be thalassemia (for example, transfusion-dependent thalassemia, thalassemia major, etc.) or thalassemia sickle cell anemia or sickle cell disease.

### Gene Therapy

A method for carrying out gene therapy in patients having various genetic and acquired diseases using a composition that increases the number of HSC in bone marrow and mobilizes these cells to the bloodstream is provided herein. In this method, HSC may be mobilized from a patient's bloodstream by administration of the composition. Peripheral blood is then collected by leukapheresis. HSC may be further enriched from the collected peripheral blood by immunoadsorption using anti-CD34 antibodies. Optionally, the enriched HSC are then expanded ex vivo by culturing them in the presence of agents that stimulate proliferation of stem cells. The enriched and optionally expanded stem cells are then transduced with an amphotrophic retroviral vector, or other suitable vectors, that expresses a gene that ameliorates the genetic or acquired disease. Optionally, the vector may also carry an expressed selectable marker, in which case successfully transduced cells may be selected for the presence of the selectable marker. The transduced and optionally selected HSC are then returned to the patient's circulating blood and allowed to engraft themselves into the bone marrow.

The invention will be more readily understood by reference to the following examples, which are included merely for purposes of illustration of certain aspects and embodiments of the present invention and not as limitations.

### EXAMPLES

### Example I

### Mice and Flow Cytometry

All experiments were performed using 8-12 week old C57BL/6 wild type mice. All mice were maintained at the UCSC Research Animal Facility in accordance with UCSC guidelines. Hematopoietic cell populations were derived from bone marrow isolated from murine femurs and tibias. Stem cell fractions were enriched using CD117 coupled magnetic beads (MACS Miltenyi). Cells were stained with unconjugated lineage rat antibodies (CD3, CD4, CD5, CD8, B220, Gr1, Mac1, and Ter119) following Goat-α-Rat PE-Cy5 (Biolegend). Stem cells were isolated using c-kit-APC-Cy7, Sca1-PB, Slamf1-PECy7, CD34-FITC, FcγrII-PE and Flk2-biotin (Biolegend) followed by streptavidin-Qdot605 (Invitrogen). Cells were sorted using a FACS Aria II (BD Bioscience). Cell populations have been defined and assessed previously (Forsberg et al., 2006). Mice were mobilized with cytoxan and G-CSF (Cy/G) as previously described (Morrison et al., 1997). Embryonic stem cells (ESC) E14 were cultured as previously described (Gaspar-Maia et al., 2009)

### Example II

### HSC In Vitro Culture and Transplantation

FACS sorted HSCs (KLS Flk2-Slamf1+) were seeded at 100-500 cells into 96-well plates and cultured with Xvivo15 (Lonza) supplemented with 25 ng/ml SCF, 5 ng/ml TPO, 50 mg/ml primocin and 55 μM β-mercaptoethanol (B-ME). Alternatively, cells were incubated on ATF024 feeder layers cells (ATCC SCRC-1007), and maintained in Dulbecco's modified Eagle medium (DMEM), 10% FCS, 100 units/ml penicillin/streptomycin, with 25 ng/ml SCF, 5ng/ml TPO. AFT024 layers were setup to 80% confluence the day before the co-culture with HSCs. G9a inhibitor UNC0638 (Sigma Aldrich) was used at 0.3 μM final concentrations and 10% dimethyl sulphoxide (DMSO) were used as the control. After 24 hours in culture cells transplanted into lethally irradiated C57BL/6 mice together with 200,000 Sca-1 depleted bone marrow cells. After 5 days in culture, cells were analyzed for cell surface markers expression by FACS, followed by transplantation into lethally irradiated C57BL/6 mice.

### Example III

### Nuclease Sensitivity Assay

FACS sorted cells were washed once with cold PBS 1% BSA and then resuspended in chromatin buffer (10 mM Tris pH 7.8, 85 mM KCl, 0.5 mM spermidine, 0.15 mM spermine, 5% sucrose, 1% BSA, 0.1% Saponin, 3 mM MgCl₂). Cell aliquots were incubated for 6 min at 37° C. with increasing concentrations of DNaseI (Whorthington) diluted in chromatin buffer. Micrococcal nuclease (Sigma Aldrich) digestion was performed using chromatin buffer plus 1 mM CaCl2. Nuclease reactions were stopped by adding equal volume of lysis buffer (10 mM Tris pH 7.8, 0.1M EDTA, 10 mM EGTA, 0.5% SDS), 0.4 mg/ml proteinase K and 20 ug/mL of RNase and incubated at 42° C. for 2 hours. DNA was isolated using phenol/chloroform, and analyzed on 1% agarose gel with SyberGold (Invitrogen). For DNA methylation assays we used the restriction enzymes MspI and HpaII, which recognize the identical DNA sequence (CCGG), but different methylation sensitivity: HpaII activity is blocked by the presence of CpG methylation. Isolated gDNA (0.5ug) was digested overnight at 37° C. with various combinations of MspI, HpaII, and HindIII; and analyzed on 1% agarose gel with SyberGold. HindIII was used as a general nuclease to increase the fragmentation size of gDNA. The methylation status of the DNA was established by measuring, for each lane, the ratio between the signal present between 1.5 kb and 5 kb, and the totality of the smear. Relative methylation was then calculated as the ratio between the values obtained for MspI and HpalI digestion for each DNA sample.

### Example IV

### Histone Western Blot and Immunohistochemistry

For western blot, FACS sorted cells were lysed in RIPA buffer, and run in a 15% gradient SDS-page (BIO-RAD). Histone modifications were assessed with antibodies against H3k27me3 (07-449 Millipore), H3 Acetylated (06-599), H4K16Ac (07-329), H3k4me3 (07-473), total H3 (06-755), H3k36me3 (Ab9050 abcam), H3k9me2 (Ab1220), H3k9me3 (Ab8898). Cells were incubated with the HDAC inhibitor trichostatin A (Cell signaling) at 37° C. for 2 hours to induce histone acetylation. For immunostaining cells were sorted onto poly-lysine coated slides, fixed with 2% PFA, permeabilized with 0.1% triton-x in PBS, and stained with H3k9me3 (Ab8898), Lamin B (sc-6217) antibodies followed by an Alexa488-Donkey-α-goat and a Alexa594-goat-α-rabbit secondary antibody (Invitrogen) plus DAPI. Images were acquired using a PerkinElmer Volocity spinning disk confocal microscope. Image analysis was done using ImageJ software (Schneider et al., 2012).

### Example V

### Electron Microscopy

For electron microscopy, cells were fixed in 2% glutaraldehyde, 1% paraformaldehyde in 0.1M sodium cacodylate buffer pH 7.4, post fixed in 2% osmium tetroxide in the same buffer, en block stained with 2% aqueous uranyl acetate, dehydrated in acetone, infiltrated, and embedded in LX-112 resin (Ladd Research Industries, Burlington, Vt.). Samples were ultrathin sectioned on a Reichert Ultracut S ultramicrotome and counter stained with 0.8% lead citrate. Grids were examined on a JEOL JEM-1230 transmission electron microscope (JEOL USA, Inc., Peabody, Mass.) and photographed with the Gatan Ultrascan 1000 digital camera (Gatan Inc., Warrendale, Pa.) at the electron microscopy facility of the Gladstone Institute (San Francisco, Calif.). Image analysis was done using ImageJ software (Schneider et al., 2012).

### Example VI

### Soft X-Ray Microscopy

FACS sorted cells were mounted in thin-walled glass capillary tubes and rapidly cryo-immobilized prior to being mounted in the cryogenic specimen rotation stage of the XM-2 Soft X-ray microscope, at the National Center for X-ray Tomography located at the Advanced Light Source of Lawrence Berkeley National Laboratory. Each dataset (i.e. 90 projection images spanning a range of 180°) was collected using a Fresnel zone plate-based objective lens with a resolution of 50 nm (Le Gros et al., 2005). The projections for every tilt were recorded using a Peltier cooled, back thinned and direct illuminated 2048*2048 pixel soft x-ray CCD camera (Roper Scientific iKon-L, Trenton, N.J., USA). Projection images were manually aligned using IMOD software by tracking fiducial markers on adjacent images (Kremer et al., 1996). The 3D X-ray tomograms were hand-segmented using Amira software (Visualisation Science Groupsm, FEI company), and used to reconstruct volumes, measure voxel values (i.e. absorption values in volume element of the reconstructed data), to calculate linear absorption coefficients (LACs) and create supplementary movies.

### Example VII

### HSPCs Chromatin Architecture is Modified Upon Differentiation

Previous studies have shown that heterochromatin abundance and distribution significantly changes upon ESC differentiation in vitro, as well as by disturbing chromatin remodelling proteins; suggesting that chromatin architecture plays a significant role in ESC maintenance and differentiation (Gaspar-Maia et al., 2009; Meshorer et al., 2006). Thus, we analyzed how does chromatin conformation changes upon differentiation of HSCs into intermediate progenitors and fully mature blood cells, using high resolution using electron microscopy (EM) and Soft X-ray tomography (SXT). EM images demonstrate that ESCs are mainly composed of euchromatin (79% of nuclear area), with a fine layer of heterochromatin at the nuclear envelope and well-defined heterochromatin foci at different positions (FIG. 1A left panel). A Significant decrease in the amount of euchromatin is observed in HSCs (KLS Flk2⁻CD150⁺), MPPs (KLS Flk2⁺CD150), and GMPs (Lin⁻cKit⁺Sca⁻CD34+Fcγ^{high}) (55%, 53% and 57% respectively) (FIG. 1B left). Finally, mature GM (CD11b⁺Gr-1⁺) and B cells (B220⁺) have the lowest amount of euchromatin as expected (34% and 37% respectively). Next, imaging by SXT allowed us to analyze the entire chromatin volume of cells, quantifying the total volumes of hetero- and euchromatin, and their distribution throughout the cell nuclei (McDermott et al., 2009). Notably, SXT is able to analyze samples in their natural hydrated conformation, therefore preventing potential artifacts in chromatin conformation due to chemical cross-linkage. Results displayed as single orthoslices shows that, similarly to EM, heterochromatin accumulates upon stem cell differentiation into progenitor cells and fully mature cells (FIG. 1A middle panel). Three-dimensional reconstruction of the cells clearly shows how hetero- and euchromatin are organized in the cell nuclei (FIG. 1A right panel). Color-coded chromatin illustrate how heterochromatin changes from a primary centric/random? position in ESCs, to a more perinuclear in HSCs, MPPs and downstream cells. Quantification of the volumes of each type of chromatin as a fraction of the nuclei demonstrates the highest percentage of euchromatin in ESCs (72.6%), followed by MPP (52.8%), GMPs (53.7%), HSCs (40.9%), B cells (37.2%) and GM cells (29.4%) (FIG. 1B right). Surprisingly, when comparing the total volumes of both fractions (in μm³), it is the euchromatin content that significantly shrinks upon differentiation rather than the heterochromatin volume that increase; which remain more or less unaltered between the different cell types (FIG. 5A). This was clearly an unexpected result, one that would not have been predicted by one of ordinary skill in the art.

This observation links to a positive correlation between nuclear size and the amount of euchromatin it contains (both by EM and SXT), which would suggests that nuclear size would be determined by euchromatin rather than heterochromatin content (FIG. 1C). Additionally, SXT allowed us to quantify the distribution of heterochromatin relative to the distance to the nuclear envelope. Within the first 0.5 μM away from the nuclear envelope ESC have the lowest amount of heterochromatin as expected, followed by HSCs, MPPs and finally mature cells which have the highest amount of heterochromatin in proximity to the nuclear envelope. Unexpectedly, GMPs values are located more close to HSC or ESC than to MPPs, suggesting that significant chromatin rearrangements might occur at this stage of differentiation, and that heterochromatin accumulation is not always linear in respect to cell lineage potential (FIG. 1D).

Next, we used a hypothetical model where: (i) cell nuclei are perfect spheres, (ii) the nuclei is divided in 2 equal parts of heterochromatin and euchromatin, (iii) and the interface between them is a regular disk (FIG. 1E); then we compared how the different cell types differ from this model. First, the interface between both chromatin fractions is significantly different to the ones expected for circular disk (value=1). The observed interphase for ESC, HSC, MPP, GMP is approximately 10-fold higher, and for GM and B cells 6-fold higher (FIG. 1F). Secondly, when we analyzed the surface of the nuclei, we observed that HSC, MPP, GMP and B cells are the closest in shape to a sphere, whereas GM cells and ESCs have a high degree of nuclei folding (FIG. 1G). The folded organization of nuclei is a known property of GM cells necessary for their function (Olins et al., 2008; Olins and Olins, 2005), whereas for ESC it might be a technical “artifact” due to the fact that ESC are squeezed into capillaries of small diameter in the process of mounting them for SXT. Overall, these results suggest that global changes in chromatin content are not required in minor transitions between adult stem cells and intermediate progenitors such as HSC, MPP and GMP, but only in large developmental steps such as, ESC to HSC, or HSC to mature cells. Also, our results illustrate that significant rearrangements in chromatin conformation occur upon ESC and HSC differentiation, and confirm that a globally open chromatin structure is not only a characteristic of ESCs, but also of adult stem and progenitor cells such as HSCs or GMPs.

### Example VIII

### HSPCs have a Globally More Open Chromatin than Mature Cells

To confirm the observations that stem cells have a global more open chromatin structure, we analysed the global degree of chromatin condensation in ESCs, HSPCs (Lin⁻Kit⁺), and mature cells (Lin⁺) by measuring their general chromatin sensitivity to DNaseI treatment. The more open the chromatin structure is the more likely it is to be accessible to DNaseI digestion. Results demonstrate that ESCs have the highest degree if sensitivity to DNaseI treatment, followed by HSPCs and then by fully mature cells (FIG. 2A). Quantification showed that HSPCs have significantly higher amounts of digested DNA at low concentrations of DNaseI compare to mature cells, which achieved the same level of DNA digestion only at higher concentrations of DNaseI. Interestingly, further separation of HSPCs into an HSC enriched fraction (KLS flk2−) and myeloid progenitors (GMPs and MEP [megakaryocyte/erythrocyte progenitors (cKit+Lin−Sca−CD34−Fcγ^{low}]); showed a higher trend in sensitivity for myeloid progenitors over HSCs, though not significant (FIG. 2A). This result would suggest that myeloid progenitors would have a more open chromatin structure than HSCs, again differing from the idea of a linear correlation between lineage potential and level of chromatin condensation. Since most HSCs are quiescent at steady state, whereas myeloid progenitors are actively proliferating, we compared proliferating HSCs (KLS flk2−) from G-CSF mobilized mice to steady-state HSCs and observed no significant differences in their sensitivity to DNaseI digestion, suggesting that cell cycle status doesn't globally affect their chromatin condensation status (FIG. 6A). We next performed the same nuclease sensitivity experiment however this time with microccocal nuclease (MNase), which induces double-strand breaks only within nucleosome linker regions. Results showed no significant differences in MNase sensitivity between HSPCs and mature cells, suggesting that higher DNaseI sensitivity in HSPCs is not due to the absence of linker histones (FIG. 2B). ESCs demonstrated significantly higher sensitive to MNase digestion, likely due to their different composition/amount of linker regions (Terme et al., 2011). Overall these results confirm that HSPCs have a global more open chromatin structure than fully differentiated blood cells.

The observed differences in chromatin condensation between ESC, HSPCs and mature cells could be due to various types of epigenetic mechanisms. DNA methylation has been shown to play important roles in HSC function, as dnmt1 and dnmt3a deficient HSC have compromised self-renewal, niche retention, and ability to give rise to multilineage hematopoiesis (Broske et al., 2009; Challen et al., 2011; Trowbridge et al., 2009). We compared the global DNA methylation levels between HPSCs and mature cells by differential digestion with restriction enzymes MspI and HpaII (Bernardino et al., 1997), however results showed no significant differences (FIG. 6b). The lack of differences is most likely due to the fact that changes in DNA methylation occur at few genomic foci and not at a global level, as shown by other recent studies (Bock et al., 2012; Ji et al., 2010). Then, we analyzed the levels of histone tail modifications in ESC, HSC/MPP enriched fraction (KLS), and mature hematopoietic cells, hypothesizing that the changes in chromatin condensation observed by DNaseI would reflect global changes in histone tail modifications levels. Results showed no significant differences in the levels of either active (H3K4me3, H3Ac, H3K36me3, H4K16Ac) or repressive (H3K27me3, H3K9me2, H3K9me3) histone marks between ESC, HSC/MPP and fully mature cells (FIG. 2C). Further separation of these populations into a HSC enriched fraction (cKit+Lin−Sca+Flk2−), myeloid progenitors (cKit+Lin−Sca−), B, T, and GM cells, also showed no significant differences in the frequency of active or repressive histone marks (FIG. 6C). Cells treatment with the HDAC inhibitor trichostatin A (TSA), significantly increased the global levels of histone acetylation demonstrating assay sensitivity (FIG. 6C). A recent study which utilized quantitative mass spectrometry as a significantly more sensitive detection method, determined that indeed there are only moderate, but significant, global differences in the levels of histone modifications between mature cells (MEFs) and induced pluripotent stem cells (IPSCs), in particular the accumulation of the repressive mark H3K9 methylation in more mature cells (Sridharan et al., 2013). These results suggest that globally more open chromatin conformation in HSPCs is not due to differences in DNA methylation or frequency of active/repressive histone modifications.

### Example IX

### Heterochromatin Mark H3K9Me3 is Redistributed Upon Differentiation

Since levels of different histone tail modifications do not appear to change in cells of very different lineage potential, we next examined whether these marks are equally distributed throughout the nucleus. In particular we focused on H3K9me3, which has been widely used as a heterochromatin mark (Gaspar-Maia et al., 2009; Meshorer et al., 2006). First we observed that H3K9me3 distribution in ESC is accumulated in well-defined foci scattered around their nucleus, as others have shown before (Gaspar-Maia et al., 2009; Meshorer et al., 2006). HSCs and MPPs H3K9me3 was preferentially localized at the nuclear periphery and mostly absent at the center of the nuclei (FIG. 3A). However upon three-dimensional analysis of confocal images we observed large number of foci of various sizes located in proximity to the nuclear envelope. Interestingly, the intermediate progenitor GMP had a less pericentric H3K9me3 distribution and more diffuse throughout the nucleus with large heterochromatin foci (FIG. 3A). Finally, mature blood cells of lymphoid and myeloid lineages had a very clear pericentric distribution of H3K9me3, which significantly overlaps with the nuclear envelope mark LaminB. Quantification of the radial distribution of H3K9me3 shows that most of heterochromatin in HSCs, MPPs, B and T cells is located at the nuclear periphery, whereas ESCs and GMPs have a wider distribution of this mark across their nuclei (FIG. 3B). H3K9me3 content in HSCs, MPPs, GM, G and T cells was mainly contained within the 25% of the nuclear area closest to the nuclear envelope, whereas only a fraction of the total heterochromatin in ESCs and GMPs was localized in this area (FIG. 3C). Overall these results show that despite having similar levels of the heterochromatin mark H3K9me3, its spatial distribution in the nucleus differs significantly, with GMPs being on particular outlier within hematopoietic cells.

### Example X

### H3K9 Methylation Through G9a is Essential for Proper HSC Differentiation

Our results indicate that condensation of euchromatin content into heterochromatin is a major process during stem cell differentiation. One particular protein involved in the transition from euchromatin into heterochromatin is the histone lysine methyltransferase G9a (Shinkai and Tachibana, 2011). G9a has been described to have a transcriptionally repressive function that depends on its activity to mono- and di-methylate H3K9 in euchromatin domains, sub sequentially recruiting other chromatin repressive complexes and promoting heterochromatin formation (Barski et al., 2007). To examine how transition from euchromatin into heterochromatin regulates HSC differentiation, we used small molecule inhibitor, UNC0638, which specifically blocks the function of G9a (Vedadi et al., 2011). We observed that in vitro culture of HSCs in the presence of UNC0638 results in a significant expansion of the total cell number as well as 4-fold enrichment in the stem cell enriched KLS fraction (FIG. 4a). The effect was concentration dependent up to 1 uM, with no significant effect on cell viability (FIG. 8a). This was clearly an unexpected result, one that would not have been predicted by one of ordinary skill in the art.

The accumulation of KLS cells upon G9a inhibition occurs in liquid culture conditions, as well as with AFT024 stromal cells as feeder layers. Global gene expression analysis of the KLS fractions of UNC0638 KLS cells as well as the DMSO control 5 days after in vitro culture revealed a significant number of HSC-related genes upregulated in UNC0638 treated cells, such as Sox6, Itga2b, Gata1 and Fgf3 (FIG. 4b). Most of these genes have significantly higher expression in freshly isolated HSCs compared to MPPs (FIG. 8b). On the other hand, we identified significantly downregulation of some genes involved in maintenance of HSC quiescence and proliferation, such as Egr1 (Min et al., 2008) and Ndn (Asai et al., 2012), as well as genes involved in stem cell engraftment, such as Robo4 (Smith-Berdan et al., 2011). We confirmed that most candidate genes identified by RNA-seq have significantly lower amount of H3K9me2 at their promoter, as well as a trend to lower amount of H3K9me3 (FIG. 4c). These results suggest that G9a is probably required for the silencing of these genes upon differentiation, and lack of functional G9a blocks/delays proper HSC differentiation. To test the functionality of this expanded KLS fraction, we test their proliferation capacity after 5 days of in vitro culture with and without UNC0638. Upon replating equal amounts of KLS from both conditions into liquid culture with SCF and TPO, UNC0638 KLS cells demonstrated equal proliferation capacity as the KLS control cells (FIG. 4d). Replating of equal amounts of Kit+ cells resulted in significant higher number of cells after 10 days in culture, as expected for their higher content of KLS cells. Surprisingly, transplantation of these cells into lethally irradiated hosts after 5 days in culture over AFT024 stromal cells, only resulted into a modest increase in the levels of engraftment for UNC0638 treated cells (FIG. 4e, 4f). Similarly, 24-hour exposure of HSCs to UNC0638 only resulted into a minor increase in engraftment after transplantation. In both scenarios, UNC0638 treated cells and control cells readout for all myeloid and lymphoid lineages in the same ratios, suggesting no defects in lineage potential (FIG. 8c). These unexpected results suggest that even though UNC0638 lead to an expansion and maintenance of KLS cells in culture; these cells may have additional pathways compromised that impair their proper engraftment upon transplantation. This was clearly an unexpected result, one that would not have been predicted by one of ordinary skill in the art.

Overall these results suggest that proper transition of euchromatin genomic regions into silenced heterochromatin, for example, by G9a function, significantly affect the development programs necessary for proper HSC maintenance and differentiation.

### REFERENCES

Adli, M., Zhu, J., and Bernstein, B. E. (2010). Genome-wide chromatin maps derived from limited numbers of hematopoietic progenitors. Nat Meth 7, 615-618.
Akashi, K., He, X., Chen, J., Iwasaki, H., Niu, C., Steenhard, B., Zhang, J., Haug, J., and Li, L. (2003). Transcriptional accessibility for genes of multiple tissues and hematopoietic lineages is hierarchically controlled during early hematopoiesis. Blood 101, 383-389.
Alabert, C., and Groth, A. (2012). Chromatin replication and epigenome maintenance. Nature Publishing Group 13, 153-167.
Ansel, K. M., Djuretic, I., Tanasa, B., and Rao, A. (2006). Regulation of Th2 differentiation and 114 locus accessibility. Annu. Rev. Immunol. 24, 607-656.
Asai, T., Liu, Y., Di Giandomenico, S., Bae, N., Ndiaye-Lobry, D., Deblasio, A., Menendez, S., Antipin, Y., Reva, B., Wevrick, R., et al. (2012). Necdin, a p53 target gene, regulates the quiescence and response to genotoxic stress of hematopoietic stem/progenitor cells. Blood 120, 1601-1612.
Attema, J. L., Papathanasiou, P., Forsberg, E. C., Xu, J., Smale, S. T., and Weissman, I. L. (2007). Epigenetic characterization of hematopoietic stem cell differentiation using miniChIP and bisulfite sequencing analysis. Proc Natl Acad Sci USA 104, 12371-12376.
Azuara, V., Perry, P., Sauer, S., Spivakov, M., Jorgensen, H. F., John, R. M., Gouti, M., Casanova, M., Warnes, G., Merkenschlager, M., et al. (2006). Chromatin signatures of pluripotent cell lines. Nat Cell Biol 8, 532-538.
Barrero, M. J., Boué, S., and Belmonte, J. C. I. (2010). Epigenetic Mechanisms that Regulate Cell Identity. Stem Cell 7, 565-570.
Barski, A., Cuddapah, S., Cui, K., Roh, T.-Y., Schones, D. E., Wang, Z., Wei, G., Chepelev, I., and Zhao, K. (2007). High-resolution profiling of histone methylations in the human genome. Cell 129, 823-837.
Bernardino, J., Roux, C., Almeida, A., Vogt, N., Gibaud, A., Gerbault-Seureau, M., Magdelenat, H., Bourgeois, C. A., Malfoy, B., and Dutrillaux, B. (1997). DNA hypomethylation in breast cancer: an independent parameter of tumor progression? Cancer Genet Cytogenet 97, 83-89.
Bernstein, B. E., Mikkelsen, T. S., Xie, X., Kamal, M., Huebert, D. J., Cuff, J., Fry, B., Meissner, A., Wernig, M., Plath, K., et al. (2006). A bivalent chromatin structure marks key developmental genes in embryonic stem cells. Cell 125, 315-326.
Bock, C., Beerman, I., Lien, W.-H., Smith, Z. D., Gu, H., Boyle, P., Gnirke, A., Fuchs, E., Rossi, D. J., and Meissner, A. (2012). DNA methylation dynamics during in vivo differentiation of blood and skin stem cells. Mol Cell 47, 633-647.
Boyer, S. W., Schroeder, A. V., Smith-Berdan, S., and Forsberg, E. C. (2011). All Hematopoietic Cells Develop from Hematopoietic Stem Cells through Flk2/Flt3-Positive Progenitor Cells. Stem Cell 9, 64-73.
Broske, A.-M., Vockentanz, L., Kharazi, S., Huska, M. R., Mancini, E., Scheller, M., Kuhl, C., Enns, A., Prinz, M., Jaenisch, R., et al. (2009). DNA methylation protects hematopoietic stem cell multipotency from myeloerythroid restriction. Nat Genet 1-11.
Challen, G. A., Sun, D., Jeong, M., Luo, M., Jelinek, J., Berg, J. S., Bock, C., Vasanthakumar, A., Gu, H., Xi, Y., et al. (2011). Dnmt3a is essential for hematopoietic stem cell differentiation. Nat Genet 1-11.
Chen, J., Odenike, O., and Rowley, J. D. (2010). Leukaemogenesis: more than mutant genes. Nat Rev Cancer 10, 23-36.
Chen, X., Skutt-Kakaria, K., Davison, J., Ou, Y. L., Choi, E., Malik, P., Loeb, K., Wood, B., Georges, G., Torok-Storb, B., et al. (2012). G9a/GLP-dependent histone H3K9me2 patterning during human hematopoietic stem cell lineage commitment. Genes Dev 26, 2499-2511.
Cui, K., Zang, C., Roh, T.-Y., Schones, D. E., Childs, R. W., Peng, W., and Zhao, K. (2009). Chromatin Signatures in Multipotent Human Hematopoietic Stem Cells Indicate the Fate of Bivalent Genes during Differentiation. Stem Cell 4, 80-93.
de Laat, W., Klous, P., Kooren, J., Noordermeer, D., Palstra, R., Simonis, M., Splinter, E., and Grosveld, F. (2008). Chapter 5 Three-Dimensional Organization of Gene Expression in Erythroid Cells. In Current Topics in Developmental Biology, J. J. Bieker, ed. (Academic Press), pp. 117-139.
Efroni, S., Duttagupta, R., Cheng, J., Dehghani, H., Hoeppner, D. J., Dash, C., Bazett-Jones, D. P., Le Grice, S., McKay, R. D. G., Buetow, K. H., et al. (2008). Global transcription in pluripotent embryonic stem cells. Cell Stem Cell 2, 437-447.
Eminli, S., Foudi, A., Stadtfeld, M., Maherali, N., Ahfeldt, T., Mostoslaysky, G., Hock, H., and Hochedlinger, K. (2009). Differentiation stage determines potential of hematopoietic cells for reprogramming into induced pluripotent stem cells. Nat Genet 41, 968-976.
Forsberg, E. C., Serwold, T., Kogan, S., Weissman, I. L., and Passegué, E. (2006). New evidence supporting megakaryocyte-erythrocyte potential of flk2/flt3+ multipotent hematopoietic progenitors. Cell 126, 415-426.
Gaspar-Maia, A., Alajem, A., Meshorer, E., and Ramalho-Santos, M. (2011). Open chromatin in pluripotency and reprogramming Nature Publishing Group 12, 36-47.
Gaspar-Maia, A., Alajem, A., Polesso, F., Sridharan, R., Mason, M. J., Heidersbach, A., Ramalho-Santos, J., Mcmanus, M. T., Plath, K., Meshorer, E., et al. (2009). Chd1 regulates open chromatin and pluripotency of embryonic stem cells. Nature 460, 863-868.
Gerlitz, G., and Bustin, M. (2010). Efficient cell migration requires global chromatin condensation. Journal of Cell Science 123, 2207-2217.
Guo, S., Zi, X., Schulz, V. P., Cheng, J., Zhong, M., Koochaki, S. H. J., Megyola, C. M., Pan, X., Heydari, K., Weissman, S. M., et al. (2014). Nonstochastic Reprogramming from a Privileged Somatic Cell State. Cell 156, 649-662.
Ho, L., and Crabtree, G. R. (2010). Chromatin remodelling during development. Nature 463, 474-484.
Ho, L., Ronan, J. L., Wu, J., Staahl, B. T., Chen, L., Kuo, A., Lessard, J., Nesvizhskii, A. I., Ranish, J., and Crabtree, G. R. (2009). An embryonic stem cell chromatin remodeling complex, esBAF, is essential for embryonic stem cell self-renewal and pluripotency. Proceedings of the National Academy of Sciences 106, 5181-5186.
Hochedlinger, K., and Plath, K. (2009). Epigenetic reprogramming and induced pluripotency. Development 136, 509-523.
Ji, H., Ehrlich, L. I. R., Seita, J., Murakami, P., Doi, A., Lindau, P., Lee, H., Aryee, M. J., Irizarry, R. A., Kim, K., et al. (2010). Comprehensive methylome map of lineage commitment from haematopoietic progenitors. Nature 1-6.
Koche, R. P., Smith, Z. D., Adli, M., Gu, H., Ku, M., Gnirke, A., Bernstein, B. E., and Meissner, A. (2011). Reprogramming Factor Expression Initiates Widespread Targeted Chromatin Remodeling. Stem Cell 8, 96-105.
Krivtsov, A. V., and Armstrong, S. A. (2007). MLL translocations, histone modifications and leukaemia stem-cell development. Nat Rev Cancer 7, 823-833.
Le Gros, M. A., McDermott, G., and Larabell, C. A. (2005). X-ray tomography of whole cells. Curr. Opin. Struct. Biol. 15, 593-600.
Lehnertz, B., Northrop, J. P., Antignano, F., Burrows, K., Hadidi, S., Mullaly, S. C., Rossi, F. M. V., and Zaph, C. (2010). Activating and inhibitory functions for the histone lysine methyltransferase G9a in T helper cell differentiation and function. Journal of Experimental Medicine 207, 915-922.
Lessard, J., and Sauvageau, G. (2003). Bmi-1 determines the proliferative capacity of normal and leukaemic stem cells. Nature 423, 255-260.
McDermott, G., Le Gros, M. A., Knoechel, C. G., Uchida, M., and Larabell, C. A. (2009). Soft X-ray tomography and cryogenic light microscopy: the cool combination in cellular imaging. Trends Cell Biol 19, 587-595.
Meshorer, E., Yellajoshula, D., George, E., Scambler, P. J., Brown, D. T., and Misteli, T. (2006). Hyperdynamic Plasticity of Chromatin Proteins in Pluripotent Embryonic Stem Cells. Dev Cell 10, 105-116.
Min, I. M., Pietramaggiori, G., Kim, F. S., Passegué, E., Stevenson, K. E., and Wagers, A. J. (2008). The Transcription Factor EGR1 Controls Both the Proliferation and Localization of Hematopoietic Stem Cells. Cell Stem Cell 2, 380-391.
Miyamoto, T., and Akashi, K. (2005). Lineage Promiscuous Expression of Transcription Factors in Normal Hematopoiesis. Int J Hematol 81, 361-367.
Morrison, S. J., Wright, D. E., and Weissman, I. L. (1997). Cyclophosphamide/granulocyte colony-stimulating factor induces hematopoietic stem cells to proliferate prior to mobilization. Proc Natl Acad Sci USA 94, 1908-1913.
Oguro, H., Yuan, J., Ichikawa, H., Ikawa, T., Yamazaki, S., Kawamoto, H., Nakauchi, H., and Iwama, A. (2010). Poised Lineage Specification in Multipotential Hematopoietic Stem and Progenitor Cells by the Polycomb Protein Bmil. Cell Stem Cell 6, 279-286.
Olins, A. L., Zwerger, M., Herrmann, H., Zentgraf, H., Simon, A. J., Monestier, M., and Olins, D. E. (2008). The human granulocyte nucleus: Unusual nuclear envelope and heterochromatin composition. Eur J Cell Biol 87, 279-290.
Olins, D. E., and Olins, A. L. (2005). Granulocyte heterochromatin: defining the epigenome. BMC Cell Biol 6, 39.
Park, I.-K., Qian, D., Kiel, M., Becker, M. W., Pihalja, M., Weissman, I. L., Morrison, S. J., and Clarke, M. F. (2003). Bmi-1 is required for maintenance of adult self-renewing haematopoietic stem cells. Nature 423, 302-305.
Schneider, C. A., Rasband, W. S., and Eliceiri, K. W. (2012). image J nat meth. Nat Meth 9, 671-675.
Shi, Y., Tae Do, J., Desponts, C., Hahm, H. S., Scholer, H. R., and Ding, S. (2008). A Combined Chemical and Genetic Approach for the Generation of Induced Pluripotent Stem Cells. Cell Stem Cell 2, 525-528.
Shin, J.-W., Buxboim, A., Spinler, K. R., Swift, J., Christian, D. A., Hunter, C. A., Léon, C., Gachet, C., Dingal, P. C. D. P., Ivanovska, I. L., et al. (2013). Contractile Forces Sustain and Polarize Hematopoiesis from Stem and Progenitor Cells. Stem Cell 1-13.
Shinkai, Y., and Tachibana, M. (2011). H3K9 methyltransferase G9a and the related molecule GLP. Genes Dev 25, 781-788.
Smith-Berdan, S., Nguyen, A., Hassanein, D., Zimmer, M., Ugarte, F., Ciriza, J., Li, D. Y., García-Ojeda, M. E., Hinck, L., and Forsberg, E. C. (2011). Robo4 cooperates with CXCR4 to specify hematopoietic stem cell localization to bone marrow niches. Cell Stem Cell 8, 72-83.
Sridharan, R., Gonzales-Cope, M., Chronis, C., Bonora, G., McKee, R., Huang, C., Patel, S., Lopez, D., Mishra, N., Pellegrini, M., et al. (2013). H3K9 and HP1 in reprogramming Nat Cell Biol 15, 1-13.
Stergachis, A. B., Neph, S., Reynolds, A., Humbert, R., Miller, B., Paige, S. L., Vernot, B., Cheng, J. B., Thurman, R. E., Sandstrom, R., et al. (2013). Developmental Fate and Cellular Maturity Encoded in Human Regulatory DNA Landscapes. Cell 154, 888-903.
Surani, M. A., Hayashi, K., and Hajkova, P. (2007). Genetic and epigenetic regulators of pluripotency. Cell 128, 747-762.
Terme, J. M., Sese, B., Millan-Arino, L., Mayor, R., Belmonte, J. C. I., Barrero, M. J., and Jordan, A. (2011). Histone H1 Variants Are Differentially Expressed and Incorporated into Chromatin during Differentiation and Reprogramming to Pluripotency. Journal of Biological Chemistry 286, 35347-35357.
Trowbridge, J. J., Snow, J. W., Kim, J., and Orkin, S. H. (2009). DNA Methyltransferase 1 Is Essential for and Uniquely Regulates Hematopoietic Stem and Progenitor Cells. Stem Cell 5, 442-449.
Vedadi, M., Barsyte-Lovejoy, D., Liu, F., Rival-Gervier, S., Allali-Hassani, A., Labrie, V.,
Wigle, T. J., DiMaggio, P. A., Wasney, G. A., Siarheyeva, A., et al. (2011). A chemical probe selectively inhibits G9a and GLP methyltransferase activity in cells. Nat Chem Biol 7, 566-574.
Waddington, C. H. (1957). The strategy of the genes. A discussion of some aspects of theoretical biology. With an appendix by H. Kacser. The Strategy of the Genes a Discussion of Some . . . ix+−262.
Weishaupt, H., Sigvardsson, M., and Attema, J. L. (2010). Epigenetic chromatin states uniquely define the developmental plasticity of murine hematopoietic stem cells. Blood 115, 247-256.
Yamamizu, K., Fujihara, M., Tachibana, M., Katayama, S., Takahashi, A., Hara, E., Imai, H., Shinkai, Y., and Yamashita, J. K. (2012). Protein Kinase A Determines Timing of Early Differentiation through Epigenetic Regulation with G9a. Stem Cell 10, 759-770.

1. A method for delaying differentiation of hematopoietic stem cells, the method comprising the steps of (i) providing a plurality of hematopoietic stem cells, wherein the hematopoietic stem cells further comprise a cKit+, Lin−, Sca1+ (KLS) fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in delaying differentiation of the hematopoietic stem cells.

2. The method of claim 1, wherein the hematopoietic stem cells are human hematopoietic stem cells.

3. The method of claim 1, wherein the suitable medium is a medium selected from the group consisting of D-MEM, N2B7, M16, F12, Roswell Park Memorial Institute (RPMI), and X-vivo15 media.

4. The method of claim 3, wherein the medium is D-MEM.

5. The method of claim 1, wherein the inhibitor of histone methyltransferase is selected from the group consisting of UNC0638, UNC0321, Bix-1294, and Chaetocin.

6. The method of claim 5, wherein the inhibitor of methyltransferase is UNC0638.

7. The method of claim 1, wherein the suitable temperature is between 30° C. and 42° C.

8. The method of claim 7, wherein the suitable temperature is 37° C.

9. The method of claim 1, wherein the incubating period is at least five days.

10. The method of claim 1, further comprising the steps of (v) providing a feeder cell population, and (vi) co-incubating the feeder cell population with the hematopoietic stem cells.

11. A method for maintaining and expanding a population of hematopoietic stem cells, the method comprising the steps of (i) providing a plurality of hematopoietic stem cells, wherein the hematopoietic stem cells further comprise a KLS fraction; (ii) culturing the hematopoietic stem cells in a suitable medium; (iii) providing an inhibitor of histone methyltransfease in the suitable medium; (iv) incubating the hematopoietic stem cells in the medium at a suitable temperature for a period of at least 24 hours; the method resulting in maintaining and expanding the population of hematopoietic stem cells.

12. The method of claim 11, wherein the hematopoietic stem cells are human hematopoietic stem cells.

13. The method of claim 11, wherein the suitable medium is a medium selected from the group consisting of D-MEM, N2B7, M16, F12, Roswell Park Memorial Institute (RPMI), and X-vivo15 media.

14. The method of claim 13, wherein the medium is D-MEM.

15. The method of claim 11, wherein the inhibitor of histone methyltransferase is selected from the group consisting of UNC0638, UNC0321, Bix-1294, and Chaetocin.

16. The method of claim 15, wherein the inhibitor of methyltransferase is UNC0638.

17. The method of claim 11, wherein the suitable temperature is between 30° C. and 42° C.

18. The method of claim 17, wherein the suitable temperature is 37° C.

19. The method of claim 11, wherein the incubating period is at least five days.

20. The method of claim 11, further comprising the steps of (v) providing a feeder cell population, and (vi) co-incubating the feeder cell population with the hematopoietic stem cells.

21-44. (canceled)

45. The method of claim 10, the method further comprising the steps of (vii) transforming at least one hematopoietic stem cell with a vector, the vector comprising a recombinant nucleic acid; (viii) incubating the transformed hematopoietic stem cell for at least 24 hours; wherein expression of the recombinant nucleic acid corrects a phenotypic defect in the hematopoietic stem cell.

46. (canceled)

47. The method of claim 10, wherein the method results in an at least four-fold enrichment of the KLS fraction of the hematopoietic stem cells.

48. The method of claim 20, the method further comprising the steps of (vii) transforming at least one hematopoietic stem cell with a vector, the vector comprising a recombinant nucleic acid; (viii) incubating the transformed hematopoietic stem cell for at least 24 hours; wherein expression of the recombinant nucleic acid corrects a phenotypic defect in the hematopoietic stem cell.

49. The method of claim 20, wherein the method results in an at least four-fold enrichment of the KLS fraction of the hematopoietic stem cells.
